SEQUENCE LISTING (12 sequences)

SEQ ID NO: 1 - DNA, 1987 bp, Mus musculus
gtactgcagaaggagaggctgggtgcgttccacactcatcatgtctgtgccacaaacaagtacatctaaagggaaagtcatgcttaaagagtacagtggacgcaagattgaagtagagcacatttttaagtggataactgcccatgcagcttctcggatcaaaactatatataatgtggagcatttgaaggaagaatggaataaaagtgatcaatactgggtaaaaatatacctatttgcaaaccttgaccaaccgccagctttcttctctgcattaagtataaaatttactggaagagttgagtttatttttgttaatgtggaaaattggaacaacaagagttatatgacagatattggtatttataacatgccatcatacatacttagaactcctgaaggaatttatagatacggaaaccacacaggtgaatttatatcccttcaggccatggattcatttctgcgctcattacaacctgaagtaaatgatctgtttgttttgagtttggttctagttaatcttatggcttggatggacttatttattacacaaggagcaaccatcaagcgatttgtggttctcataagcactttagggacatacaattccctattaattatttcttggctacctgtgttgggctttctacagctcccttacttagatagcttttatgaatatagtttaagattgctgcgatattctaatacaaccacactggcttcgtgggtaagggcagactggatgttttactcttcacacccagccctgtttctcagtacataccttggacatggtttgctaattgattactttgagaagaagagacgtcgcagcaacaatgatgaagttaatgcgaataatttagaatggttatcaagtctgtgggactggtacaccagctacctcttccacccgattgcttcttttcagaactttcctgtagactctgattgggacgaagaccctgacttattcttggaacggttagctttccctgacctttggcttcaccctctgataccaactgattatattaaaaacttaccaatgtggcgatttaaatgtcttggggttcagtctgaagaagaaatgtcagagagttctcaagacactgaaaatgactcggatagtgacaacatggacacttttagtagtagtaaggatatatttgaagataaacaaagcgttgttcacagttctccaggaagaacaagtcactgtgatactgaggcttgttcatgtgccaataaatgtgagagcagcccatgtgaaaggaagaggaggtcgtatggatcacataatactgatgaagatatggaaccggactggttaacttggcctgcgggtacgctgcactgtactgaatgtgttgtttgccttgagaattttgaaaatggatgtttgctaatggggttgccttgtggtcacgtgtttcaccagaattgcattgttatgtggttggctgggggccgacactgttgccctgtttgtcgttggccttcatataagaaaaagcagccatatgcacaacaacagccgttgtcaaatgatgttccatcttaaccatgtgcaatttgtccttaataagccttgagtatcttacagcttgcctttttaatgttagtcacaatgtttttgtggtttgaagtttagtttaatgttagtgcagtgacaggaaatacacattatgctgatgttgatgacagaatttatttggttgccttgtgtgtcaattgaatgcatactaaactgtaaaaaaaatttatttacagcattgaaaattcagaagttaatggttttttgtaagcacaaaagaagtatggtagaaattaatcttaacaagactttatgaggcaggatcaaatcctagtgggcctgagcaggtttcttaccctaagtgttttctccctttttacaatctctgtccagcacctcttggttaaataatgtatgctctgagacatgaaattaaaacagatc

SEQ ID NO: 2 - DNA, 1680 bp, Mus musculus
atcaagcttatcgataccgtcgatcgacggtatcgataagcttgatatcgaattcctgcagccggccgcacgtctaagaaaccattattatcatgacattaacctataaaaataggcgtatcacgaggccctttcgtcttcaagaattccgatcatattcaataacccttaatataacttcgtataatgtatgctatacgaagttattaggtctgaagaggagtttacgtccagccaagcttaggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggtcgaaaggcccggagatgaggaagaggagacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagccaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaattgacctgcaggggccctcgaggggtcgacggtatcgataagcttgat

SEQ ID NO: 3 - DNA, 1987 bp, Mus musculus; identical to SEQ ID NO: 1

SEQ ID NO: 4 - DNA, 31 bp, Mus musculus
gatctataaaataaattattttaaaagcaga

SEQ ID NO: 5 - DNA, 10148 bp, Mus musculus
aggttgtaggtttctgatcttaagctgtggtaaggtgactggtttttgtaggtgttttgctggattactgatttctcgggtaattgcaacgtgattttgagatggagggagaagaaatggaatccttttgctgacactggctatgagtaatggtggtctacctcacagcctaatatcttggataggaaagaattttatagggggctttgaaaaatcagtacttagcatattcatctaagtaaaaacaactataactttttagtcacacaaggattaccttgtatgtttaactgactttttattttgtaagtttttagtggtagaggaaagccgagcatatagatagtagaaaaaatatctttctctttcgtgtaccatatttgtattttattacatttactttagcaccttttattacaattttaatacttcttctttttccctctcagtcagctgaaagtaaacttgaatcatgatgctttgcccgtaaacagtcaccacattttctgtgaatgtcccccataccctctcccccactcccctacccacccattcccactttttggccctggcgttcccctgtactggggcatataaggtttgcgtgtccaatgggcctctctttccagtgatggctgactaggccatcttttgatacatatgcagctagagacacgagctccggggtactggttagttcatattgttgttccatctataggattgcagaccccattagctccttgggtactttctctagctcctccattgggggccctgtgatccatccaatagctgactgtgagcatccacttctgtgtttgctaggcccctgcatagtctcacaagagacagctatatcagggtcctttcagcaaacgcttgctagtgtatgcaatggtgtcatcgtttggaggctaattatgggatggctccctggatatggcagtctctagatgatccatccttttgtctcagctccaaactttgtctctgttactccttccatgggtgattgtttccaattctaagaaggggcaaagtgtccacactttggtctttgttcttcttcagtttcgtgtgttttacaaattgtctcttatatttcgggtatactaagtttctgggctaatatccacttatcagtgagtacatatcatttgagttcttttgtgattgtgttacctcactcaggatgatgccctccaggtccaaccatttgcctaggaatttcataaattcattctttttaatagctgagtagtgaacccttgacatttaatacctaatatttgttccttgtcccataaaagtctttatatctctgtctttgttttgttgacttatctgttgggtttagtttccctttcttccatcttcccttagttcctgtagagaaactgctgttcatgtgtgtgtgtgtgttgtgctaaggcttgaattcaggacctcagcataataggcaagtaccccaagctacaccccagccctctctaaattttttaattttgagatagtctcactagattgcctgagttggtcttgaactcattatagcttaggtaagccttgagttctcaatcttcctgcatcagcctcttagattacatgcctgagttgcaagcctgatgcctgttctggtactaatatttttaacagtacaaattggcttatatagtgttccttgttttctttcaagattaaatttgagtgctgctcatatacatcgtatcaggatgcatgtagtgtcagttctgttgttggtgattcgaaatgtgtttaatatctgtcaagtctttcctttgtgagataccttttccctctgtaatttaagaagcaacctgtgattctttgtgatcttttccctcatctcagtggttttaacagccagtgatgattctttgacttggtcatcaagttagaatatggtcattttcaaatttctgcttaattgcacatttattagctggctgtgtgtgtgagagagaggcgaggagagctttctatttttctcccatgttttgaatatcactggacttagatgtttatcattattattattattattattattattattattattattacccagtgtattaaaactttaggtgctagagagaatgctcgttggttaagagcactggctactcttacaggagacctgggtttgattcccagcatccatatgatagccaactagccaccatcttatttgtaactacagtttcaggaggtctaatacatttcttgcctctgtaggcagtgaacacacaaggtacacaggcaaaacacccatgtaagtaagtaagtaagtaagtaagaaagacagtcagtcagtcagtcagtcccaaagcactcataaaaattattaaattttaaaaaatgcttatttttggtactcagtgtcatacttagctagtggaaacctctttacactagccttatgtcttgttactaatgtcttaatcctgaagcacttgtttgctttttgaccctggtttcttgtcatctttttttcagagtacccaagtttcatgatggacacctctaagatccttagttagctgcactccaaaatggaaatgggaaaaacaaaatataaattatttccaggatactaacaattttttcttgcatgataaattgtgccttactttcaaatactaatagaaaataggtgctttttcacttttagtgggattatgtcataataaatccatcataaattgaaagtttcacaagtaaaacatgcatttacaagtctaaagtcccaactttacgtagaaaaatgtcggttatccttcaaggtcctctgactggcagttccagcttgatacctctatccagaatactgtactttcctaggctgaacagtgaaattctagtcagaatttcaactaactacctctaaagagacagacttctttgtcatcatcctaaaaattgagaaaccatcaaacatcagcagaatgcttgtctaaatagagaattcataaagccctgaattggtcctcagcaccatataaactgacatggtggtgcatacctgtcgtcccactcagaggtggaggatcaagggtccagttcatgttcaactacatagagagttcaaaactaacctgagagatattagaccttgtcttaaataggaaaacacctagtagaaaggactagcagctatggattgtatttcataaatttgagaacttaaaataactgcagccattgctgcatatacttctgaacaagcgctgaaatcctttccttgtaaatggagtcccaagatttctcacctcttcttttgtccatgctttcagcagatggttctcaaatttacctgttcttcatgggctctttccacagcaccagttttatacacagatttctactagactcgcttgaagtacttcggatcgaacttcggtcttttggggcatactaggcaagcactctcccatcctcagcctttgatttttgtagcttgggatggccttgaactcataatcatcctgaattcaattctgccatgccctggctctgggttcttttaattaactacagtgctctgttgtctttcttcatccccagtcttccttttaaggtcttaatggagataggtggagttacaaaatgtacatgaaagtgtagaaatgtttagacaaaatttctctagctttccagagcacatcacacattggatatcattatcctaaacagaccttttgaatcttttccattcataatccctttcattcaagaaggtggctttggggcatgtaggtatataaaatagctacacatatcagacatttactggcaataaaattgtaaatatatttaaaagcctctgctatccctacagctttgctgttcactaaagagtaagacagtttttcatactagtaagataagtgtgcttatttttacataaagaattaaatcttggggttggagagatggctcaacagttaagagcactgactattcttccagggctcttaagttcaattctcagcaaccacatagtggctcacaaccatctgtaatgggatccaatgcccggatctgttgtgtctgaagacagctacagtgtactcatgcacataaataaatctttaaaaaaaaagaattaaatcttagccaagtgtagtggtacacacctctaatccaagggaggcagaggcaggtggatcttcctgagtttgaggccaacctggtctataataaagtcccagaacagacagggctgttacataaagaaaccctgccttgaaaaagtcaaaacaaccccaaaataatcttgtctgaatattttatgttagcctagaagcccagcacatctttgacattatttagaactgatcaacatttttatttttatgattgtcagtgcagaagatgcaactttgtataaatctgtaatataaaatgaaaaaaaagcagaaatatgtttagagccatttcagaaattgttgaaaattctgcctaatcccatgtcaaaattgatttaacagtttttaaggaaactgagtcagcaactggcagtttttaaagaatctgatacaatccagtctaaacagttgctagctatactagtatactgatatggtagttacatgctaagaaatgacaataaagaaaaattttaagcttttgctttctgtgattaaaccattatgtttctgtaagagcagaaaactgtatatacacttaaaatactgcagttcaccacgtgatagcgttgagtctgagccaagatgtttcaggcagtttgtgttggcactgtgtggcatccctacacgtgaagtgcagcatgtgtgttgtatgttcagagacaaggctgtggtgaagccaggctgtgaaacacaggtgaatgttaccagaaacaccttagattcatcatctgtctgattctgtattacctttgttcttttgcaaattcaggaagtttttactcttaaaacatttatccagacccccaaatttagttactggaccaagttgaggactagacaacaaagaaatcagcattggccatcaatggtccctctcatttctggcttactatttaggctcattttcatttcacattctttttttttaagacttttatttatttattatatgtgagtacactgtagctgtcttcagacactctggatgagggtgtcagatcttgttacagatggttgtgagccaccatgtggttgctgggatttgaactcaggacctttggaagagcagtcgatgttcttaaccactgagccatctcaccagccctcatttcacattcttaccacccgtgttatttttcctccggtgggtgctttgttgtcatcccctctacctggttgtattgttcctttttctactctttctgttggcttggttgaattttctccctaagttctgcgccttcccatcttctgcttccttactagacttaaaagggggcaaagatacttacaggtagtgacagtcagtgaaggacaccttattcaagtgattcaactaatgtcatcaacagctgagtggagagatgtgtgctctatgtttcaggtactttggacattcctgatagatgcattgaactaaacattgcatgtgcctttttagatcctttatggtagggcctgtgtgttattaatctagctacccacaatgcatggtccaaaatggacaaaatacttttttcagtgttatgtgtgcacaaaaattcctcagtgaaaaaacaaaattacttcaaaatggttagagggactgccagggtttcgggtcttcatacacgtggctcatcccttaattgttaaatctgctcttagcagattttccctgaggtagagaactgaaggaaatactgaagattgatagattttatttcaacttgtttttaaagaaaagttctgtttctttgaacagtttcttgtatttaaggaaccccaagcatgtggtctttgcaggtgcaggattccctggataatatgacacataatttgttaggagacttttgaaacctaaattattttgtgtgtaaaatctcttcattctgtaaagttgataagctccctgaggggtgaaatattatgtagaattgcctcctctcagtatgtaagatcctatatagatacagcaaataattgctggcttgaatcttaagaacatattttagttttacctctctcccatctcatgtaggtaaggaaggtaaaagatgctatttactttttaggttacttgggagcctggcaagttattcttgtaaaatctttctcttttctcacctgtgttttcattttattattccttaaaactcagtcaaaggagaaggctggagagatggtgcagaggttgagtgcactggctgcttttacagaggacctgggttcaattcctggcctcacatggtgtctcacaaacatctgtaactcagtttcagggaatctgattctggtttctggcctctttgggatccagggatgcatgcagtacacagacatacacgcaggcaaaacatccatacatgttcaaaacaaaacaaagcaactatgccaaaagagcataaaagcaagtaaaatgtctggctgattttaaattaagacaattaatttaaaattagttaagacaattaatttaaagtgctgaaaattaattgttaaaaatgatattttaaaaattaatttccttttttttttttaattgtaaaaactacatctgtgtatttcctgggaggggcaggacacttgccctggcacttgtggaagctcgagatgatgtgcaggaattcgttctcttctcccaccgtgtgggtcccagggatgcagtgcagattcatcagcatcctggcaggcacctttatgtgctgagccacctcaccacccatcctgtgtgttgggatcatcctgtttctcttcttggttgtcactaattgtatgttggtactggcagtctattcaggtgttttactctctgacctaagttcccattcaggctgggaatgtaactcactaataggatacagcagaccctggattccggctgtgtgaaggacatttcccttcataatatcctctgcagtgttggaataggcagcatgcttacttaatagacgatcacaaattcaggacagttgagattaagatgagagtaggacacagctttcaaacctacgtgtcctcagggctcgagtgcagagtggtgcagctatggtatctatgtgtgttttcacctttttttgttttgttttgttttttgttttttgagacagggtttctctgtgtagccctggctgtcctggaacttactctgtagaccaggctggcctcaaactcagaaattcgcctgcctctgcctcccgagtgcttgggttttcacttttaagtatggtcaaaagaatgtatcagtggatccactctgacccatggcttttaggcagagttgtacctttctagcttgttcatggatctccctgccatattcactctcaaaaataaactctcaggaaaagctatgggaatttggactggagatctagctcagaggtagaacacttgcatgcgcgcgcgcgcgcgcgcgcacacacacacacacacacacacactaaagccctgtaggcaccagatgacataaaattgactttcataacagtgtctatagagtaagtaccagcatcattgctattacaaaggtctgcaagctcttttatttgggggcatatttttctttttcttccttttcttttttataattgatcagtcagatttatatatcaataaattctcagttcatgagatatccacacaatatgttcagagccgatgataatgatacggggtggggagagtgcttatttttcggaatgttttctcttcgttgacaattcagggactagtgatatttctacataggtagcttgattggagttttgtggtttgtttttgtttttttttcagaaagaccaataggcacggtgttctacagcaaagaatggttagactactggtattgttcccatttgtttttacttaattcctgagccaagtggccaaacagtaggtttctgaaacaaaatccatcaaactaattcctgcctttgttgtcagatacttggataagagaaccctcttaaaataccaaacagcgacatcctatgccttaaattttgtgtacaaataatttctagttctgaatgtggtacacttgtgacccatctcaatggatgctgctttatttagaatccacctagtactgtttgacaaggggtagaaaagaatgtgactcttcaccgtttttctatttaaaatcatcagaatcatgtgtgatagtcactcagtccagtctttctgccttaacttaggttactagtggccttgtagtacctggcacagtcatcatctaacaaaagacatggcacccttacaagatctataaacattctacccaccaggatccacagagaagcacgggattagctggcaacacctcagcctctggtgttgaaggtgggcacttcatttggatacttctcatgccaccatctttgggaaggaggatgagtcagaggtcatcttttgaaaaatctggtcctgttccaatcattgtcttggaaatacatttccatcacatatcaaaatagtagctatttcaccgggatctttatttcaagaaagcctgtaagctcatttctgtgctttcagcactaaattcatcccctcaccctgaacatctccacaagcaggagcaggagcctcgctcagggcagcttctcactccaggcaagcaggttcctgctgatactgcatgagggcagagttacagtgtcagctgttttcctggctgtgtaaaggatggaggggaatatgccaaagcaaaaggcaattcttagcacaagatgcaccacatggagttctacagaaactggagaaaagacattacaattttctagggggcggaacaacccacaggaacccaccgacttgctttcctttaccctaagtgggattggagcaaataaactcactcaggtatttagtgtctgttcaaatagcttcaggtgtttagcagaggaggtcatgttaaatataaattacatgtttatctgagtccacaatctgagtgcagccctagtgtgtcctgaagtaatagtcctagagaaagaacagtgggtggggtgtgggaaggacttttagatgccctgtaccatactgagacaagaaaggggtggagcaaatagacagtattcttccatctttaaccagttccagacagcaaagaggaatagtaggccaggaagatgacttggtggctaaaagcacttgcagggtaagcaggacaacctaggtttggattcagagaacccacgtaaaagccaaatgcagttcgcgcacatttgtaatcccaacactcctgtagtcctgaagcccattgaccagctagactagagtgcacactgcagcagcagagacaacagagaccctgccttaacaggcaggaagtgaaaataccctctgactgggttctggttcccgacatgcacgatatggtatgcatcttagtttcttctcttgcttcaaagaaacaccatgactagggcaacatattaaaaacagcattttattgaaggcttgtttacagcttcagaaggttagtctatggccatcatggtggagaacatgacattaggcaggcaggcatggcactaggacagtagcttagaacttacatgttatccacagcatcagacaggatagcaagactgggtcttgtatgggcttttgaaacctcaaagcccatcctccaatgcgtgacacacctcctgcaataaggctgtaccactaacccttcctaaacagatgaccaactaggaaccaaacacacattttgaggcctatggggattatcctcattcaaacccccacagtatgtttgcctgtacagacagacagatacatacagacataccccaaaaccaaacctacactactaccaaacaaaacaaaacaaaacaaaattggaaggaaaatatgtttatcttttcctcttaggttattcctttgaaagacaatgtgtttccataaaaacaagaatcgggtgctgaagagatggtgatgtgattaaaagcacatattatactgctctttcaaaagatc

SEQ ID NO: 6 - DNA, 20248 bp, Artificial Sequence
  vector
acggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgagcttggcccagtgccaagctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcccctcgaggccgctctagggccgcatctagctagagtcgatcgaccagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgttaatagatcatcaatttctgcatcctcgactctagtggatctgcattccaccactgctcccattcatcagttccataggttggaatctaaaatacacaaacaattaggaatcagtagtttaacacattatacacttaaaaattttatatttaccttagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggcgaggacctgcagggtcgctcggtgttcgaggccacacgcgtcaccttaatatgcgaagtggacctgggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgactagaactaattcttatcgtatttcatttttgattaaatttagattttgtgtgtgtgtatgtgtgtgtgtgtagaggagtatatatgctcatctgagtgcaggtgccattgaagtccagaagatggtgtaggataccttggatctggagtcataggcagctatgagcctcccagtgtaaacactgatttaaaaaaaaaaatcaacatttttatatagccaaatgaactgatgacaaagctgggtaaaatggtatatgcctgtattcccagtggcaggcagaatgggaggtctcaaattctaagccaatctatataatgaaacgctagcacaaaacaaagctctgaaaaatgttttcttgatagtatcattgaatttattctgacctttaaagtgttctttttgaattggcagtaaaagattttatgttgatacatggtaatataaattttttaaaaacaatatatgcatgtagtcttgtattcattgaagatactttagaataatatttagagaaatatatattagtaacccagtatttaataagcattagagattttttccccaaacatggaaaacattatttaatttgacaattataacaggaaatgttcttttgggtagtctcaagttctttagaataacaagacttaaaaataccagttctgaactgaacagaaaacataaaaggaggaaatgagaatattaaagcttacctgttctacaaagcaagatttcgtgtcataaagatgtcatttgatttatgacacaactcagaagccagtgtagttataagagaaaagataaaaagaatgaccaaaagtagagaagtgggactggggagatgtttcagcaatgaagaatattttctgatcttgcagagagaatatgctctgttaagggactgaatcaacacagtagaagcagagtcaggagctatgattcaaagtcactaatatagcttcccagagtcctcaacaacctgctctttaaaagacgacatgccatcttcatttatctcaaattacaccaccgcattcctgagataaattagtcatgttgccattataaaacatatgccacataactagacttcctctctggatcctatgaatgtgtaacatactcatctaaccccaataaaatatgaaatgtgcttagtaaataaaatgtaacatcccttatatcagtattctgaagcaacataagaaatcaagtggtgggctggagagatggctcagtgattaagagcaccggctgctcttccaaaggtcgtgagttcaaatcccagcaaccacatggtggctcacaaccatccataatgagatctgacgacctcttctggtgcatctgaagacagctacagtgtacttagatacaataataaataaatctttaaaaaaaaaaaaagaaatcaagtggtatccattctcaagtctatagcaaaacatatctacatggtagacttttttactttttatcagataattttatatgtcataaaattctacatggcaggttttaggaagaatttttaaaaatgtctttagttatctatccataagtaacgtgatggatgttttagtttttggtatttcttatcacctgttttttcagaggtgacaaatgtgatatcaagaacttgctggggaccaaaaggagtagagtcactgaccttgtccataaatgtacacaggtagtgattataattatgctcttctccttcaccccatcagtaaactgaattggcctgaaactctttgtatactttctgtctttgttttctaagtgttaaatcgttgtttttatggtactcattaaagagaaaaggaaggataactgtaacttgaagtcgctgtcttttggattggcttatgtgaagatcactttttagtagttagcgcctatcgcactgtggtgttaggatcaagcttatcgataccgtcgatcgacggtatcgataagcttgatatcgaattcctgcagccggccgcacgtctaagaaaccattattatcatgacattaacctataaaaataggcgtatcacgaggccctttcgtcttcaagaattccgatcatattcaataacccttaatataacttcgtataatgtatgctatacgaagttattaggtctgaagaggagtttacgtccagccaagcttaggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggtcgaaaggcccggagatgaggaagaggagacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagccaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaattgacctgcaggggccctcgaggggtcgacggtatcgataagcttgatccttgtgagagtacttagttgagaagcggtaaggtaggtgcctatcaaggactctgaaccatctcagggactcagcatgtaggagcattcatctctttctctgtttctctctaatttctaaccatcttttaaggccaagttcttctccaaacctttattgagctattcacctgctcaaattcctgatgatatctgttctttgatctttctttactcttcctttgatactggtttcacatccataacaggagttttaaattccatatgagcagaaattacttcccattatctttggatcttagtatgtaatgcagtggcacataagagaggctaaagtattttctgactctttggcttaggtacagcttatacagaattttttcttttcttaaacacagaaatttaaagacgactacagtgaacttgcttgttctggcacagttgccaagtattggtctctctttggttacttgaagattatctggtctgagagaagagggttttaaaagggccctaaagaaaactagtagaacgtaagttgttcaacagcctcgctgttcattcagttcactctcttacaggtactgcagaaggagaggctgggtgcgttccacactcatcatgtctgtgccacaaacaagtacatctaaagggaaagtcatgcttaaagagtacagtggacgcaagattgaagtagagcacatttttaagtggataactgcccatgcagcttctcggatcaaaactatatataatgtggagcatttgaaggaagaatggaataaaagtgatcaatactgggtaaaaatatacctatttgcaaaccttgaccaaccgccagctttcttctctgcattaagtataaaatttactggaagagttgagtttatttttgttaatgtggaaaattggaacaacaagagttatatgacagatattggtatttataacatgccatcatacatacttagaactcctgaaggaatttatagatacggaaaccacacaggtgaatttatatcccttcaggccatggattcatttctgcgctcattacaacctgaagtaaatgatctgtttgttttgagtttggttctagttaatcttatggcttggatggacttatttattacacaaggagcaaccatcaagcgatttgtggttctcataagcactttagggacatacaattccctattaattatttcttggctacctgtgttgggctttctacagctcccttacttagatagcttttatgaatatagtttaagattgctgcgatattctaatacaaccacactggcttcgtgggtaagggcagactggatgttttactcttcacacccagccctgtttctcagtacataccttggacatggtttgctaattgattactttgagaagaagagacgtcgcagcaacaatgatgaagttaatgcgaataatttagaatggttatcaagtctgtgggactggtacaccagctacctcttccacccgattgcttcttttcagaactttcctgtagactctgattgggacgaagaccctgacttattcttggaacggttagctttccctgacctttggcttcaccctctgataccaactgattatattaaaaacttaccaatgtggcgatttaaatgtcttggggttcagtctgaagaagaaatgtcagagagttctcaagacactgaaaatgactcggatagtgacaacatggacacttttagtagtagtaaggatatatttgaagataaacaaagcgttgttcacagttctccaggaagaacaagtcactgtgatactgaggcttgttcatgtgccaataaatgtgagagcagcccatgtgaaaggaagaggaggtcgtatggatcacataatactgatgaagatatggaaccggactggttaacttggcctgcgggtacgctgcactgtactgaatgtgttgtttgccttgagaattttgaaaatggatgtttgctaatggggttgccttgtggtcacgtgtttcaccagaattgcattgttatgtggttggctgggggccgacactgttgccctgtttgtcgttggccttcatataagaaaaagcagccatatgcacaacaacagccgttgtcaaatgatgttccatcttaaccatgtgcaatttgtccttaataagccttgagtatcttacagcttgcctttttaatgttagtcacaatgtttttgtggtttgaagtttagtttaatgttagtgcagtgacaggaaatacacattatgctgatgttgatgacagaatttatttggttgccttgtgtgtcaattgaatgcatactaaactgtaaaaaaaatttatttacagcattgaaaattcagaagttaatggttttttgtaagcacaaaagaagtatggtagaaattaatcttaacaagactttatgaggcaggatcaaatcctagtgggcctgagcaggtttcttaccctaagtgttttctccctttttacaatctctgtccagcacctcttggttaaataatgtatgctctgagacatgaaattaaaacagatcatcgaattcctgcagccggaacccttaatataacttcgtataatgtatgctatacgaagttattaggtccctcgaagaggttcactagtactgggtacccgatctataaaataaattattttaaaagcagaaggttgtaggtttctgatcttaagctgtggtaaggtgactggtttttgtaggtgttttgctggattactgatttctcgggtaattgcaacgtgattttgagatggagggagaagaaatggaatccttttgctgacactggctatgagtaatggtggtctacctcacagcctaatatcttggataggaaagaattttatagggggctttgaaaaatcagtacttagcatattcatctaagtaaaaacaactataactttttagtcacacaaggattaccttgtatgtttaactgactttttattttgtaagtttttagtggtagaggaaagccgagcatatagatagtagaaaaaatatctttctctttcgtgtaccatatttgtattttattacatttactttagcaccttttattacaattttaatacttcttctttttccctctcagtcagctgaaagtaaacttgaatcatgatgctttgcccgtaaacagtcaccacattttctgtgaatgtcccccataccctctcccccactcccctacccacccattcccactttttggccctggcgttcccctgtactggggcatataaggtttgcgtgtccaatgggcctctctttccagtgatggctgactaggccatcttttgatacatatgcagctagagacacgagctccggggtactggttagttcatattgttgttccatctataggattgcagaccccattagctccttgggtactttctctagctcctccattgggggccctgtgatccatccaatagctgactgtgagcatccacttctgtgtttgctaggcccctgcatagtctcacaagagacagctatatcagggtcctttcagcaaacgcttgctagtgtatgcaatggtgtcatcgtttggaggctaattatgggatggctccctggatatggcagtctctagatgatccatccttttgtctcagctccaaactttgtctctgttactccttccatgggtgattgtttccaattctaagaaggggcaaagtgtccacactttggtctttgttcttcttcagtttcgtgtgttttacaaattgtctcttatatttcgggtatactaagtttctgggctaatatccacttatcagtgagtacatatcatttgagttcttttgtgattgtgttacctcactcaggatgatgccctccaggtccaaccatttgcctaggaatttcataaattcattctttttaatagctgagtagtgaacccttgacatttaatacctaatatttgttccttgtcccataaaagtctttatatctctgtctttgttttgttgacttatctgttgggtttagtttccctttcttccatcttcccttagttcctgtagagaaactgctgttcatgtgtgtgtgtgtgttgtgctaaggcttgaattcaggacctcagcataataggcaagtaccccaagctacaccccagccctctctaaattttttaattttgagatagtctcactagattgcctgagttggtcttgaactcattatagcttaggtaagccttgagttctcaatcttcctgcatcagcctcttagattacatgcctgagttgcaagcctgatgcctgttctggtactaatatttttaacagtacaaattggcttatatagtgttccttgttttctttcaagattaaatttgagtgctgctcatatacatcgtatcaggatgcatgtagtgtcagttctgttgttggtgattcgaaatgtgtttaatatctgtcaagtctttcctttgtgagataccttttccctctgtaatttaagaagcaacctgtgattctttgtgatcttttccctcatctcagtggttttaacagccagtgatgattctttgacttggtcatcaagttagaatatggtcattttcaaatttctgcttaattgcacatttattagctggctgtgtgtgtgagagagaggcgaggagagctttctatttttctcccatgttttgaatatcactggacttagatgtttatcattattattattattattattattattattattattattacccagtgtattaaaactttaggtgctagagagaatgctcgttggttaagagcactggctactcttacaggagacctgggtttgattcccagcatccatatgatagccaactagccaccatcttatttgtaactacagtttcaggaggtctaatacatttcttgcctctgtaggcagtgaacacacaaggtacacaggcaaaacacccatgtaagtaagtaagtaagtaagtaagaaagacagtcagtcagtcagtcagtcccaaagcactcataaaaattattaaattttaaaaaatgcttatttttggtactcagtgtcatacttagctagtggaaacctctttacactagccttatgtcttgttactaatgtcttaatcctgaagcacttgtttgctttttgaccctggtttcttgtcatctttttttcagagtacccaagtttcatgatggacacctctaagatccttagttagctgcactccaaaatggaaatgggaaaaacaaaatataaattatttccaggatactaacaattttttcttgcatgataaattgtgccttactttcaaatactaatagaaaataggtgctttttcacttttagtgggattatgtcataataaatccatcataaattgaaagtttcacaagtaaaacatgcatttacaagtctaaagtcccaactttacgtagaaaaatgtcggttatccttcaaggtcctctgactggcagttccagcttgatacctctatccagaatactgtactttcctaggctgaacagtgaaattctagtcagaatttcaactaactacctctaaagagacagacttctttgtcatcatcctaaaaattgagaaaccatcaaacatcagcagaatgcttgtctaaatagagaattcataaagccctgaattggtcctcagcaccatataaactgacatggtggtgcatacctgtcgtcccactcagaggtggaggatcaagggtccagttcatgttcaactacatagagagttcaaaactaacctgagagatattagaccttgtcttaaataggaaaacacctagtagaaaggactagcagctatggattgtatttcataaatttgagaacttaaaataactgcagccattgctgcatatacttctgaacaagcgctgaaatcctttccttgtaaatggagtcccaagatttctcacctcttcttttgtccatgctttcagcagatggttctcaaatttacctgttcttcatgggctctttccacagcaccagttttatacacagatttctactagactcgcttgaagtacttcggatcgaacttcggtcttttggggcatactaggcaagcactctcccatcctcagcctttgatttttgtagcttgggatggccttgaactcataatcatcctgaattcaattctgccatgccctggctctgggttcttttaattaactacagtgctctgttgtctttcttcatccccagtcttccttttaaggtcttaatggagataggtggagttacaaaatgtacatgaaagtgtagaaatgtttagacaaaatttctctagctttccagagcacatcacacattggatatcattatcctaaacagaccttttgaatcttttccattcataatccctttcattcaagaaggtggctttggggcatgtaggtatataaaatagctacacatatcagacatttactggcaataaaattgtaaatatatttaaaagcctctgctatccctacagctttgctgttcactaaagagtaagacagtttttcatactagtaagataagtgtgcttatttttacataaagaattaaatcttggggttggagagatggctcaacagttaagagcactgactattcttccagggctcttaagttcaattctcagcaaccacatagtggctcacaaccatctgtaatgggatccaatgcccggatctgttgtgtctgaagacagctacagtgtactcatgcacataaataaatctttaaaaaaaaagaattaaatcttagccaagtgtagtggtacacacctctaatccaagggaggcagaggcaggtggatcttcctgagtttgaggccaacctggtctataataaagtcccagaacagacagggctgttacataaagaaaccctgccttgaaaaagtcaaaacaaccccaaaataatcttgtctgaatattttatgttagcctagaagcccagcacatctttgacattatttagaactgatcaacatttttatttttatgattgtcagtgcagaagatgcaactttgtataaatctgtaatataaaatgaaaaaaaagcagaaatatgtttagagccatttcagaaattgttgaaaattctgcctaatcccatgtcaaaattgatttaacagtttttaaggaaactgagtcagcaactggcagtttttaaagaatctgatacaatccagtctaaacagttgctagctatactagtatactgatatggtagttacatgctaagaaatgacaataaagaaaaattttaagcttttgctttctgtgattaaaccattatgtttctgtaagagcagaaaactgtatatacacttaaaatactgcagttcaccacgtgatagcgttgagtctgagccaagatgtttcaggcagtttgtgttggcactgtgtggcatccctacacgtgaagtgcagcatgtgtgttgtatgttcagagacaaggctgtggtgaagccaggctgtgaaacacaggtgaatgttaccagaaacaccttagattcatcatctgtctgattctgtattacctttgttcttttgcaaattcaggaagtttttactcttaaaacatttatccagacccccaaatttagttactggaccaagttgaggactagacaacaaagaaatcagcattggccatcaatggtccctctcatttctggcttactatttaggctcattttcatttcacattctttttttttaagacttttatttatttattatatgtgagtacactgtagctgtcttcagacactctggatgagggtgtcagatcttgttacagatggttgtgagccaccatgtggttgctgggatttgaactcaggacctttggaagagcagtcgatgttcttaaccactgagccatctcaccagccctcatttcacattcttaccacccgtgttatttttcctccggtgggtgctttgttgtcatcccctctacctggttgtattgttcctttttctactctttctgttggcttggttgaattttctccctaagttctgcgccttcccatcttctgcttccttactagacttaaaagggggcaaagatacttacaggtagtgacagtcagtgaaggacaccttattcaagtgattcaactaatgtcatcaacagctgagtggagagatgtgtgctctatgtttcaggtactttggacattcctgatagatgcattgaactaaacattgcatgtgcctttttagatcctttatggtagggcctgtgtgttattaatctagctacccacaatgcatggtccaaaatggacaaaatacttttttcagtgttatgtgtgcacaaaaattcctcagtgaaaaaacaaaattacttcaaaatggttagagggactgccagggtttcgggtcttcatacacgtggctcatcccttaattgttaaatctgctcttagcagattttccctgaggtagagaactgaaggaaatactgaagattgatagattttatttcaacttgtttttaaagaaaagttctgtttctttgaacagtttcttgtatttaaggaaccccaagcatgtggtctttgcaggtgcaggattccctggataatatgacacataatttgttaggagacttttgaaacctaaattattttgtgtgtaaaatctcttcattctgtaaagttgataagctccctgaggggtgaaatattatgtagaattgcctcctctcagtatgtaagatcctatatagatacagcaaataattgctggcttgaatcttaagaacatattttagttttacctctctcccatctcatgtaggtaaggaaggtaaaagatgctatttactttttaggttacttgggagcctggcaagttattcttgtaaaatctttctcttttctcacctgtgttttcattttattattccttaaaactcagtcaaaggagaaggctggagagatggtgcagaggttgagtgcactggctgcttttacagaggacctgggttcaattcctggcctcacatggtgtctcacaaacatctgtaactcagtttcagggaatctgattctggtttctggcctctttgggatccagggatgcatgcagtacacagacatacacgcaggcaaaacatccatacatgttcaaaacaaaacaaagcaactatgccaaaagagcataaaagcaagtaaaatgtctggctgattttaaattaagacaattaatttaaaattagttaagacaattaatttaaagtgctgaaaattaattgttaaaaatgatattttaaaaattaatttccttttttttttttaattgtaaaaactacatctgtgtatttcctgggaggggcaggacacttgccctggcacttgtggaagctcgagatgatgtgcaggaattcgttctcttctcccaccgtgtgggtcccagggatgcagtgcagattcatcagcatcctggcaggcacctttatgtgctgagccacctcaccacccatcctgtgtgttgggatcatcctgtttctcttcttggttgtcactaattgtatgttggtactggcagtctattcaggtgttttactctctgacctaagttcccattcaggctgggaatgtaactcactaataggatacagcagaccctggattccggctgtgtgaaggacatttcccttcataatatcctctgcagtgttggaataggcagcatgcttacttaatagacgatcacaaattcaggacagttgagattaagatgagagtaggacacagctttcaaacctacgtgtcctcagggctcgagtgcagagtggtgcagctatggtatctatgtgtgttttcacctttttttgttttgttttgttttttgttttttgagacagggtttctctgtgtagccctggctgtcctggaacttactctgtagaccaggctggcctcaaactcagaaattcgcctgcctctgcctcccgagtgcttgggttttcacttttaagtatggtcaaaagaatgtatcagtggatccactctgacccatggcttttaggcagagttgtacctttctagcttgttcatggatctccctgccatattcactctcaaaaataaactctcaggaaaagctatgggaatttggactggagatctagctcagaggtagaacacttgcatgcgcgcgcgcgcgcgcgcgcacacacacacacacacacacacactaaagccctgtaggcaccagatgacataaaattgactttcataacagtgtctatagagtaagtaccagcatcattgctattacaaaggtctgcaagctcttttatttgggggcatatttttctttttcttccttttcttttttataattgatcagtcagatttatatatcaataaattctcagttcatgagatatccacacaatatgttcagagccgatgataatgatacggggtggggagagtgcttatttttcggaatgttttctcttcgttgacaattcagggactagtgatatttctacataggtagcttgattggagttttgtggtttgtttttgtttttttttcagaaagaccaataggcacggtgttctacagcaaagaatggttagactactggtattgttcccatttgtttttacttaattcctgagccaagtggccaaacagtaggtttctgaaacaaaatccatcaaactaattcctgcctttgttgtcagatacttggataagagaaccctcttaaaataccaaacagcgacatcctatgccttaaattttgtgtacaaataatttctagttctgaatgtggtacacttgtgacccatctcaatggatgctgctttatttagaatccacctagtactgtttgacaaggggtagaaaagaatgtgactcttcaccgtttttctatttaaaatcatcagaatcatgtgtgatagtcactcagtccagtctttctgccttaacttaggttactagtggccttgtagtacctggcacagtcatcatctaacaaaagacatggcacccttacaagatctataaacattctacccaccaggatccacagagaagcacgggattagctggcaacacctcagcctctggtgttgaaggtgggcacttcatttggatacttctcatgccaccatctttgggaaggaggatgagtcagaggtcatcttttgaaaaatctggtcctgttccaatcattgtcttggaaatacatttccatcacatatcaaaatagtagctatttcaccgggatctttatttcaagaaagcctgtaagctcatttctgtgctttcagcactaaattcatcccctcaccctgaacatctccacaagcaggagcaggagcctcgctcagggcagcttctcactccaggcaagcaggttcctgctgatactgcatgagggcagagttacagtgtcagctgttttcctggctgtgtaaaggatggaggggaatatgccaaagcaaaaggcaattcttagcacaagatgcaccacatggagttctacagaaactggagaaaagacattacaattttctagggggcggaacaacccacaggaacccaccgacttgctttcctttaccctaagtgggattggagcaaataaactcactcaggtatttagtgtctgttcaaatagcttcaggtgtttagcagaggaggtcatgttaaatataaattacatgtttatctgagtccacaatctgagtgcagccctagtgtgtcctgaagtaatagtcctagagaaagaacagtgggtggggtgtgggaaggacttttagatgccctgtaccatactgagacaagaaaggggtggagcaaatagacagtattcttccatctttaaccagttccagacagcaaagaggaatagtaggccaggaagatgacttggtggctaaaagcacttgcagggtaagcaggacaacctaggtttggattcagagaacccacgtaaaagccaaatgcagttcgcgcacatttgtaatcccaacactcctgtagtcctgaagcccattgaccagctagactagagtgcacactgcagcagcagagacaacagagaccctgccttaacaggcaggaagtgaaaataccctctgactgggttctggttcccgacatgcacgatatggtatgcatcttagtttcttctcttgcttcaaagaaacaccatgactagggcaacatattaaaaacagcattttattgaaggcttgtttacagcttcagaaggttagtctatggccatcatggtggagaacatgacattaggcaggcaggcatggcactaggacagtagcttagaacttacatgttatccacagcatcagacaggatagcaagactgggtcttgtatgggcttttgaaacctcaaagcccatcctccaatgcgtgacacacctcctgcaataaggctgtaccactaacccttcctaaacagatgaccaactaggaaccaaacacacattttgaggcctatggggattatcctcattcaaacccccacagtatgtttgcctgtacagacagacagatacatacagacataccccaaaaccaaacctacactactaccaaacaaaacaaaacaaaacaaaattggaaggaaaatatgtttatcttttcctcttaggttattcctttgaaagacaatgtgtttccataaaaacaagaatcgggtgctgaagagatggtgatgtgattaaaagcacatattatactgctctttcaaaagatcgagtcgaccattgcggccgccaccgcggtggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaactccgtcga

SEQ ID NO: 7 - DNA, 3261 bp, Mus musculus
cggctacagcgggtccagtgctctgggggcgacggccgcggcctgcagaggcggcagggacggtggccgcggttggcgcgcgcgtccgcacggggctgggcaccgcgcggctacgcagctgacaggaagccgcccgggagagccgcgttggggcctagcgttatttgctttttcctctttttttcctcccttcacgactgtcgtctcgcgctcctccgcagcgggagccgccgcgacgccccctcgcgggccgcggggcctgataggcgccgcccgcgggaacctggagctgccgccaggcctgggcggccgccggggcctgaagcctgggcgttcggcgcggcgctgcggcggtcactccaacccgcgctgggcgcgccgggccccgggcctggcccagccgagtaccgcgccctctggaccactcagtccggccccaaccgctctccgctggcccgcagtccctcggagctgcctcccggtcaccccaggtgggctcttgccctcttccagccgtccgcagctcgatgggtggggtgcgtttgaacgtccccccttctccctttctcagctcctgaccgcaaggccagagccggggctctcagtcgcacgcacgccgcgatccacctgcctccccgcggggatggcccgccggtgaccgccgcgcgccctcgctgccctcgcccaggctgcgctctgtcgccccgcactcgatccccttcccttgtccacggcgtcccggctcctggcgacgccaagagggcgaagatgtggctgaagctgtttttcttgctcctgtatttcctggtcctgttcgtcctggccaggttttttgaggccattgtgtggtacgagactggcatctttgctactcagctggtagatccggtggcattgagcttcaagaagctgaagaccattctggagtgtcgagggctgggctactccgggctgccggagaagaaagatgttcgggtgctggtggagaagtcaggtgacttgatggaaggtgaactctattctgctcttaaggaagaagaggcatctgaatctgtttctagtaccaatttcagtggtgaaatgcatttctatgagcttgtggaagacacaaaagatggcatctggctggttcaggtcatagcaaatgacagaagtcctttggtgggtaaaatccactgggagaaaatggtgaaaaaagtgtcaagatttggaatacggacaggcactttcaactgttccagtgatcccaggtactgcagaaggagaggctgggtgcgttccacactcatcatgtctgtgccacaaacaagtacatctaaagggaaagtcatgcttaaagagtacagtggacgcaagattgaagtagagcacatttttaagtggataactgcccatgcagcttctcggatcaaaactatatataatgtggagcatttgaaggaagaatggaataaaagtgatcaatactgggtaaaaatatacctatttgcaaaccttgaccaaccgccagctttcttctctgcattaagtataaaatttactggaagagttgagtttatttttgttaatgtggaaaattggaacaacaagagttatatgacagatattggtatttataacatgccatcatacatacttagaactcctgaaggaatttatagatacggaaaccacacaggtgaatttatatcccttcaggccatggattcatttctgcgctcattacaacctgaagtaaatgatctgtttgttttgagtttggttctagttaatcttatggcttggatggacttatttattacacaaggagcaaccatcaagcgatttgtggttctcataagcactttagggacatacaattccctattaattatttcttggctacctgtgttgggctttctacagctcccttacttagatagcttttatgaatatagtttaagattgctgcgatattctaatacaaccacactggcttcgtgggtaagggcagactggatgttttactcttcacacccagccctgtttctcagtacataccttggacatggtttgctaattgattactttgagaagaagagacgtcgcagcaacaatgatgaagttaatgcgaataatttagaatggttatcaagtctgtgggactggtacaccagctacctcttccacccgattgcttcttttcagaactttcctgtagactctgattgggacgaagaccctgacttattcttggaacggttagctttccctgacctttggcttcaccctctgataccaactgattatattaaaaacttaccaatgtggcgatttaaatgtcttggggttcagtctgaagaagaaatgtcagagagttctcaagacactgaaaatgactcggatagtgacaacatggacacttttagtagtagtaaggatatatttgaagataaacaaagcgttgttcacagttctccaggaagaacaagtcactgtgatactgaggcttgttcatgtgccaataaatgtgagagcagcccatgtgaaaggaagaggaggtcgtatggatcacataatactgatgaagatatggaaccggactggttaacttggcctgcgggtacgctgcactgtactgaatgtgttgtttgccttgagaattttgaaaatggatgtttgctaatggggttgccttgtggtcacgtgtttcaccagaattgcattgttatgtggttggctgggggccgacactgttgccctgtttgtcgttggccttcatataagaaaaagcagccatatgcacaacaacagccgttgtcaaatgatgttccatcttaaccatgtgcaatttgtccttaataagccttgagtatcttacagcttgcctttttaatgttagtcacaatgtttttgtggtttgaagtttagtttaatgttagtgcagtgacaggaaatacacattatgctgatgttgatgacagaatttatttggttgccttgtgtgtcaattgaatgcatactaaactgtaaaaaaaatttatttacagcattgaaaattcagaagttaatggttttttgtaagcacaaaagaagtatggtagaaattaatcttaacaagactttatgaggcaggatcaaatcctagtgggcctgagcaggtttcttaccctaagtgttttctccctttttacaatctctgtccagcacctcttggttaaataatgtatgctctgagacatgaaattaaaacagatctataaaataaattattttaaaagc

SEQ ID NO: 8 - DNA, 23 bp, Artificial Sequence
  primer
aacttgaagtcgctgtcttttgg

SEQ ID NO: 9 - DNA, 24 bp, Artificial Sequence
  primer
gaatgggctgaccgcttcctcgtg

SEQ ID NO: 10 - DNA, 24 bp, Artificial Sequence
  primer
tcttggttaaataatgtatgctct

SEQ ID NO: 11 - DNA, 23 bp, Artificial Sequence
  primer
aacttgaagtcgctgtcttttgg

SEQ ID NO: 12 - DNA, 24 bp, Artificial Sequence
  primer
cccctataaaattctttcctatcc

(19) United States
(12) Patent Application Publication
(10) Pub. No.: US 2010/0050277 A1
(43) Pub. Date: Feb. 25, 2010

(54) KNOCKOUT ANIMAL EXHIBITING ANXIETY-LIKE BEHAVIOR

(75) Inventors: Tamotsu Hashimoto, Kyoto (JP); Atsushi Tsujimura, Kyoto (JP)

Correspondence Address: MERCHANT & GOULD PC, P.O. BOX 2903, MINNEAPOLIS, MN, 55402-0903, US

(73) Assignee: Tamotsu HASHIMOTO, Kyoto-shi, Kyoto (JP)

(21) Appl. No.: 12/515,064

(22) PCT Filed: Nov. 16, 2007
(86) PCT No.: PCT/JP2007/072257

§ 371 Date: Jun. 8, 2009

(30) Foreign Application Priority Data
Nov. 17, 2006 (JP) 2006-312039

(51) Int. Cl. G01N 33/50; A01K 67/027; C12N 15/85; C12N 15/74

(52) U.S. Cl. 800/3; 800/13; 800/21; 435/320.1

(57) ABSTRACT

A vector for creating kf-1 gene knockout nonhuman animals exhibiting increased anxiety-like behaviors, and containing Lox-pM-M-kf-1[in3b]-kf-1[ex4a]-LoxP,

(wherein, M is a selection marker gene, pM is a promoter for the expression of the selection marker gene, kf-1[in3b] is a sequence represented by SEQ ID NO: 2, and kf-1[ex4a] is a sequence represented by SEQ ID NO: 3); kf-1 gene knockout nonhuman animals exhibiting increased anxiety-like behaviors produced by using the vector or a descendant of the animal, and a method for use of the same.

### TECHNICAL FIELD

The present invention relates mainly to a knockout nonhuman animal exhibiting anxiety-like behaviors and the usage thereof.

### BACKGROUND ART

The kf-1 gene is highly expressed in the hippocampus and the cerebellum of a healthy human brain but barely expressed in the cerebral cortex. The present inventors identified two genes, which are expressed more frequently in the cerebral cortex of an Alzheimer's disease (AD) patient. One of the two genes was novel at that time. The novel gene was named kf-1 (Non-Patent Document 1, Patent Document 1, etc.).

The other gene was the gene for glial fibrillary acidic protein (GFAP), which was conventionally known being expressed more in the cerebral cortex of Alzheimer's disease patients.

In order to clarify the function of kf-1 gene, the present inventors attempted to create a knockout mouse using the Cre-lox conditional expression system.

Thereafter, another group (Department of Psychiatry, School of Medicine, Showa University) identified a gene having an enhanced expression in the cerebral cortex after chronic administrations of selective-serotonin-reuptake-inhibitor (SSRI), which is an antidepressant drug, and reported that the identified gene was an orthologue of the human kf-1 gene (Non-Patent Document 2). The group also reported that antidepressive repeated electroconvulsive treatment showed similar augmentation (Non-Patent Document 3).

Patent Document 1: Japanese Unexamined Patent Publication No. 9-215495

Non-Patent Document 1: Yasojima et al., 1997, BBRC, 231(2):481-487

Non-Patent Document 2: Yamada, M., et al., 2002, BBRC, 78(1):150-157

Non-Patent Document 3: Nishioka et al., 2003, J. Neural. Transm., 110(3):277-285

### DISCLOSURE OF THE INVENTION

### Problem to be Solved by the Invention

An object of the present invention is to provide a nonhuman knockout animal that is useful for the elucidation of anxiety-like behavior, or the development of medicine for preventing, treating or alleviating anxiety-like behavior, and the method for using the animal.

### Means for Solving the Problem

The present inventors conducted extensive research on the behavioral aspects of kf-1 knockout mice in order to elucidate the possible association of kf-1 gene to neurological diseases.

Specifically, the present inventors constructed a vector having loxP sites in the kf-1 gene, and subjected 432 clones of resulting G418-resistant ES cells to PCR-based screening analysis. Consequently, five homologous recombinant clones were obtained and chimeric mice were generated by using these ES cells. Thereafter, they were mated with Cre-expressing mice to produce kf-1 null mice, and an analysis mainly of the effect on the nervous system was conducted.

As a result, increased anxiety-like behaviors were observed in anxiety-like behavior tests. The present invention has been accomplished by conducting extensive research based on this finding.

Specifically, the present invention provides the followings:

Item 1: A vector for creating a kf-1 gene knockout nonhuman animal exhibiting anxiety-like behaviors comprising:

LoxP-pM-M-kf-1[in3b]-kf-1[ex4a]-LoxP

wherein M is a selection marker gene,

pM is a promoter for expression of the selection marker gene,

kf-1[in3b] is a base sequence represented by SEQ ID NO: 2, and

kf-1[ex4a] is a base sequence represented by SEQ ID NO: 3.

It is preferable that the pM-M in the vector of Item 1 be pgk-Neo, which is a pKJ2-derived neomycin resistance gene.

Preferably, the vector according to Item 1 has a base sequence represented by SEQ ID NO: 1 and/or SEQ ID NO: 4 located outside the regions flanked by a set of LoxP cassette.

Preferably, the vector according to Item 1 consists of a base sequence represented by SEQ ID NO: 6.

Preferably, a use of a vector comprising:

LoxP-pM-M-kf-1[in3b]-kf-1[ex4a]-LoxP

wherein M is a selection marker gene,

pM is a promoter for expression of the selection marker gene,

kf-1[in3b] is a base sequence represented by SEQ ID NO: 2, and

kf-1[ex4a] is a base sequence represented by SEQ ID NO: 3;

for creation or generation of a kf-1 gene knockout nonhuman animals exhibiting anxiety-like behaviors.

Item 2: A kf-1 gene knockout nonhuman animal (may be male or female) created by using the vector of Item 1 or a descendant of the animal.

Preferably, a kf-1 gene knockout nonhuman animal (may be male or female) or a descendant thereof for use in screening of compounds for their potential to prevent, treat or alleviate an anxiety disorder, or anxiety-like behaviors as a part of depression or other psychiatric diseases.

Item 3: A modified animal derived from the kf-1 gene knockout nonhuman animal or a descendant thereof of item 2, or a descendant of the modified nonhuman animal.

Preferably, a modified nonhuman animal lacking kf-1 gene or a descendant thereof or a descendant of the modified nonhuman animal, wherein the kf-1 gene knockout nonhuman animal (may be male or female) is usable for screening compounds or their potential to prevent, treat or alleviate an anxiety disorder, or anxiety-like behaviors as a part of depression or other psychiatric diseases, particularly, the genuine anxiety disorder.

Item 4: A method for screening compounds for their potential to prevent, treat or alleviate an anxiety disorder or anxiety-like behaviors as a part of depression or other psychiatric diseases,

the method comprising Steps (1) to (3) below:

(1) administering a test compound to the nonhuman animals or descendants thereof of Item 2 or 3;

(2) observing or measuring the anxiety-like behaviors of the nonhuman animal group before and after the administration of the test compound or observing or measuring anxiety-like behaviors of the nonhuman animal group to which the test compound is administered and that of a placebo administration group; and

(3) comparing the results in Step (2) to select a test compound that can decrease anxiety-like behaviors.

The method of Item 4 for screening compounds for their potential to prevent, treat or alleviate anxiety-like behaviors,

preferably having Steps (1) to (3) below:

(1) administering a test compound or placebo to the nonhuman animals or descendants thereof of Item 2 or 3;

(2) observing anxiety-like behaviors of the nonhuman animal group to which the test compound is administered and that of the group to which the placebo is administered; and

(3) comparing the results in Step (2) to select a test compound that can suppress anxiety-like behaviors.

Two similar groups using wild-type mice may also be added to the above steps.

Item 5: A medical composition for preventing, treating or alleviating an anxiety disorder or anxiety-like behaviors that is a part of depression or other psychiatric diseases,

the medical composition comprising the compound selected for the first time by the screening method of Item 4 as an active ingredient.

Among the medical composition of Item 5, a composition for preventing, treating or alleviating, in particular, an anxiety-like behaviors, which contains the compound selected by the screening method of Item 4 as an active ingredient.

The present invention is explained in detail below.

### 1. Vector

The vector of the present invention is desirably usable for creating a knockout nonhuman animal exhibiting anxiety-like behaviors.

The structure of the vector is LoxP-pM-M-kf-1[in3b]-kf-1[ex4a]-LoxP,

wherein M is a selection marker gene,

pM is a promoter for the selection marker gene expression,

kf-1[in3b] is a base sequence represented by SEQ ID NO: 2, and

kf-1(ex4a) is a base sequence represented by SEQ ID NO: 3.

Here, the kf-1[in3b] is a part of the intron 3 of a mouse kf-1 gene. The kf-1[ex4a] is a large part of the exon 4 of the mouse kf-1 gene. The LoxP denotes the LoxP sequences used for a Cre-LoxP system.

The selection marker gene and the promoter for its expression may be suitably chosen from known ones. A preferable example thereof is the pgk-driven neomycin (G418) resistance gene, which is derived from pKJ2, resulting in pgk-Neo.

The vector in the present invention may have a kf-1 intron or exon, or a part thereof, outside of the region flanked by the LoxP cassettes that are inserted at the both ends. For example, the vector may have base sequences represented by SEQ ID NO: 1 and/or SEQ ID NO: 4.

One example of the preferable vectors of the present invention includes a vector consisting of the base sequences represented by SEQ ID NO: 6.

As long as the effect of the present invention can be maintained, the gene can be modified appropriately or a suitable linker may be added to the vector of the present invention.

The vector of the present invention may be suitably constructed by a known method, for example, the method described in Example 1.

By using the vector of the present invention with the Cre-LoxP system, a kf-1 gene knockout animal exhibiting anxiety-like behaviors can be reliably produced.

### 2. Knockout Nonhuman Animals or Its Descendants

In the present invention, the term of “knockout nonhuman animals” includes any animals other than humans. A usable animal is not limited to the vertebrates. Examples of the animals include mouse, rat, rabbit, guinea pig, swine, sheep, goat, etc. Among these, a mouse is preferably used in the present invention. A “descendant” animal can be generated by the standard method such as mating using the knockout nonhuman animals as parents or ancestors.

The present invention is explained in detail below using a mouse as a sample animal.

### 2-1. Creation of Knockout Nonhuman Animals

The knockout nonhuman animals of the present invention can be created using the above-mentioned target vector with the Cre-LoxP system. Specifically, the vector is produced by inserting a kf-1 gene or a part thereof between two LoxP sequences. The thus-obtained target vector is introduced into ES cells. Resistant clones can be obtained by culturing cells introduced with the target vector in the presence of an appropriate drug, for example, G418. Thereafter, desired clones having homologous recombination are selected by, for example, southern blot analysis, and microinjected into pregnant mice with early embryos to obtain a chimeric mouse. The thus-obtained chimeric mouse is crossed with the wild-type mouse to obtain a heterozygous mouse. The thus-obtained heterozygous mouse is mated with another heterozygous mouse similarly obtained to obtain a homozygous mouse.

Specifically, the knockout nonhuman animal of the present invention can be produced by, for example, the method disclosed in Example 1.

### 2-2. Characteristics

The knockout nonhuman animal of the present invention or a descendant thereof exhibits anxiety-like behaviors.

In the present specification, the term “anxiety-like behaviors” means behaviors associated with anxiety.

In the present invention, the term “exhibiting anxiety-like behaviors” indicates that, in behavioral anxiety tests, mice exhibit remarkably increased anxiety-like behaviors than the wild-type or normal mice (hereunder, they are referred to as the control group or control mice). In particular, it means that the mice display significantly increased anxiety-like behaviors but do not exhibit significant differences from the control group in general locomotor abilities or other behavioral activities, learning ability, depression-like behaviors defined by depression model experiments such as the forced swim test, and social activities defined by the social behavior test.

“Depression” model experiments mentioned above include, for example, the forced swim test and/or tail suspension test. Examples of anxiety-like behavior tests include the light/dark transition test, and the elevated plus-maze test and/or the startle-response test (prepulse inhibition test).

The experimental results of the knockout nonhuman animals of the present invention in the forced swim test, which is one of the “depression” model tests, were negative, i.e., there is no significant difference from the results of the control group. However, in the light/dark test and/or elevated plus-maze test, which are anxiety-like behavior tests, remarkably enhanced anxiety-like behaviors are observed, i.e., the knockout nonhuman animal of the present invention exhibits significantly increased anxiety—but not depression-like behaviors than the control group. The knockout nonhuman animals of the present invention exhibit an elevated sensorimotor gating ability in startle response test (prepulse inhibition test), which examines the control of sensory-information filtering. An animal or human suffering from schizophrenia is defective in sensorimotor gating ability to filter out unnecessary sensory-information.

However, the knockout mice do not exhibit such a symptom as observed in schizophrenic animals and humans, but exhibit the increased ability for suppressing the startle response in the prepulse inhibition test than the wild-type mice do.

This seems to be probably because suppression of the startle response may be relevant to anxiety-like behaviors triggered by an increase in sensitivity to sensory information.

Such characteristics of the knockout mice of the present invention indicate that they can be used as an animal model for some human mental disorders, for example, social withdrawal disorder, which is pointed out to be one of the depressive symptoms and a heralding symptom or complication of Alzheimer-type dementia, although the detailed entity of these symptoms are not yet clarified.

Specifically, the knockout nonhuman animal of the present invention is usable as a model animal for research to elucidate the control mechanisms of anxiety-like behaviors including behavioral disorders that are considered to consist of multiple symptoms of depression, for example, anxiety, decreased libido, social withdrawal, social isolation, suicidal ideation, etc. The knockout nonhuman animal of the present invention is also usable as an experimental animal for screening compounds for potential to prevent, treat or alleviate anxiety-like behaviors.

The knockout nonhuman animal of the present invention or a descendant thereof has, for example, the following characteristics:

### Genetic Classification Targeted Mutation Congenic

Origin of the strain: The mouse was created by the introduction of DNA having LoxP both before and after the exon 4 of the mouse kf-1 gene to mouse embryonic stem cells. The resulting mouse was mated with a Cre Tg mouse, resulting in a mouse and descendants thereof in which the kf-1 exon 4 was deleted.
Microbiological Breeding Environment: conventional
Microbiological Characteristics: ICLAS (microscopic examination I, culture I, serum I) negative, S. aureus positive
Details of the strain (characteristics and use): Exhibiting anxiety-like behaviors similar to symptoms observed in “social withdrawal”
Breeding and Mating: Reproductive efficiency A
Remarks: Abnormal behaviors were observed. Anxiety-like behaviors or behaviors similar to that observed in symptoms of “social withdrawal disorder” was exhibited in the light/dark test and elevated plus-maze test. However, “depression-like” symptoms defined by the forced swim test and/or tail suspension test were not observed.
In contrast with schizophrenia, the kf-1 knockout mice exhibit significantly increased ability of sensorimotor gating and increased inhibition of the startle response compared to the wild-type mice.

The present invention includes the knockout nonhuman animal produced in the manner described above or its modified descendant. The modified animals include any one that is modified by genetic manipulation, mating, etc., or a descendant thereof.

### 3. Screening Method

The present invention provides a method for screening compounds, by using the knockout nonhuman animal, which are useful for prevention, treatment or alleviation of anxiety disorders or anxiety-like behaviors that constitute a basic disorder of depression or other psychiatric diseases.

Specifically, the present invention provides a method for screening compounds, using the knockout nonhuman animal, which are useful for the prevention, treatment or alleviation of an anxiety disorder or anxiety-like behaviors that constitute a basic disorder of depression or other psychiatric diseases. The method includes the following Steps (1) to (3).

Step (1): Administering the test compound to the nonhuman animals or descendants thereof;

Step (2): Observing and measuring the anxiety-like behaviors of the nonhuman animals in the group to which the test compound was administered and the group to which a placebo was administered; and

Step (3): Selecting a test compound that can alleviate anxiety-like behaviors by comparing the results of the observation and measurement in Step (2).

There is no limitation to the administration method of the test compound and a standard method can be employed.

There is no limitation to the means for measuring anxiety-like behaviors, and the measurement can be conducted by an anxiety-like behavior test using a known method.

Examples of anxiety-like behavior tests include the light/dark test, elevated plus-maze test, prepulse inhibition test, etc.

The light/dark test is conducted according to the following procedure. A mouse is first placed in a dark compartment of the light-dark boxes having a dark compartment and light compartment communicably adjacent to each other, followed by measurements, within a predetermined time, of (1) the duration of time that the mouse stays in the light compartment and the duration of time that the mouse stays in the dark compartment; (2) the number of transition times the mouse traveled between the two compartments; (3) the latency time until the mouse enter the light compartment for the first time; (4) the total distance traveled in respective compartments; etc.

It is determined that anxiety-like behaviors increase if the time spent in the light compartment is shortened, the number of times traveled between the compartments decreases, or the latency time before going into the light compartment for the first time is prolonged.

The elevated plus-maze test is conducted in the following procedure. A cross-shaped elevated maze (having two open arms without walls and two closed arms with walls) is prepared. A mouse is placed on the platform in the crossing center of the maze. Measurements are conducted within a predetermined time regarding (1) the duration time that the mouse stays in the open arms and the duration time that the mouse stays in the closed arms; (2) the respective number of times that the mouse enters the open and closed arms; (3) the total traveling distance on the respective arms; etc.

If the time spent in the open arms is prolonged, it is determined that the anxiety-like behaviors are decreased. In contrast, if the stay time in the open arms is shortened, it is determined that the anxiety-like behaviors are increased.

The prepulse inhibition test is conducted in the following procedure. Mice are placed in a test box equipped with a device for measuring a change in load for detecting a startle reaction. When the mice become acclimated to the surroundings of the device, the duration of white noise is used in the box for adaptation of mice to the new atmosphere. The magnitude of the startle responses are expressed as an increase in load when a strong acoustic stimulus is given suddenly, or when strong acoustic stimulus is given after the prepulse stimulus, i.e., A and B successively. The reduction ratio of the startle response (prepulse inhibition) C (%) can be obtained by C=100(1−B/A).

When the load resulting from a response to strong acoustic stimulus decreases compared to that resulting from a preceding acoustic stimulus, i.e., when the value of C increases, it is assumed that the startle response is reduced. It is known that the reduction in the startle response is significantly lower in a schizophrenic group compared to that of the control group, probably because the schizophrenic group has disorders in sensorimotor gating. If the reduction of the startle response is significantly higher than that of the control group, they have reinforced sensorimotor gating, i.e., it reflects the condition where the anxiety-like behaviors are increased.

Regarding this behavioral test, if the knockout nonhuman animals being administered the test compound exhibit alleviation or prevention of anxiety-like behaviors compared to the group to which a placebo was administered, the test compound can be identified as a compound or a candidate compound that is useful for prevention, treatment or alleviation of an anxiety disorder or anxiety-like behaviors that are a part of depression or other psychiatric diseases.

Examples of anxiety disorders associated with psychiatric diseases other than anxiety disorder associated with depression, include anxiety disorders attributable to schizophrenia, dementia, social withdrawal disorder, etc., but not limited to these.

### 4. Medical Composition

The medical composition of the present invention may contain a compound selected by the above-explained screening method as an active ingredient.

In particular, the present invention provides a medical composition that contains, as an active ingredient, the compound that was selected according to the above-described screening method. The medical composition of the present invention can prevent, treat or alleviate an anxiety disorder or anxiety-like behaviors that are a part of depression or other psychiatric diseases.

The medical composition obtained by the present invention may contain appropriate pharmacological carriers, various excipients generally blended with a medicinal composition, and other medicinal properties as long as they do not adversely affect the effects of the present invention.

There is no limitation to the production method of the medical composition of the present invention; it can be appropriately produced in accordance with a publicly known procedure.

The medical composition obtained by the present invention contains, as an active ingredient, the compound that was selected for the first time by the above-described screening method. The medical composition can prevent, treat or alleviate an anxiety disorder or anxiety-like behaviors that are a part of “depression” or other psychiatric diseases. Hereunder, the mouse “depression” symptom that is determined by the forced swim test and/or tail suspension test is expressed using quotation marks, i.e., “depression”, so as to distinguish it from human depression that is clinically diagnosed.

Specifically, the pharmacological compounds obtained by the present invention can be used for preventing, treating or alleviating an anxiety disorder or anxiety-like behaviors as one of the symptoms of depression such as fear, decreased libido, social withdrawal, social isolation, suicidal ideation and behavioral disorders resulting thereof, which are caused by emotional drive seen generally in depression.

### EFFECT OF THE INVENTION

Currently, it is becoming clear that there is a large difference based on gender in humans at the onset of the symptoms of “social withdrawal disorder” (about 80% withdrawers are male), and many suffering from symptoms of “social withdrawal disorder” have relatives with a similar medical history, etc. Therefore, it is believed that a biological factor may be involved in the onset of the symptoms of “social withdrawal disorder”.

In order to develop a medicine that is effective for alleviating the symptoms of “social withdrawal disorder”, it is necessary to use a simple animal (e.g., mouse) that exhibits the symptoms of “social withdrawal disorder” and like anxiety disorders, but the presence of such an animal has not been reported yet.

The kf-1 null mouse developed in the present invention does not exhibit the symptoms of “depression” that are defined by the forced swim test and/or the tail suspension test. However, the kf-1 null mice of the present invention specifically exhibit anxiety-like behaviors defined by the elevated plus-maze test and the light/dark test. In other words, the kf-1 null mouse of the present invention exhibits behaviors similar to that observed in “social withdrawal” symptoms.

These characteristics of social withdrawal-like behaviors are believed to be some of the symptoms of depression, a prodrome of Alzheimer's disease, etc. However, they may be associated with complications of various psychoneuroses of which details have not been substantively elucidated, for example, anxiety disorder, decreased libido, social isolation, suicidal ideation and like behavioral abnormalities.

Therefore, the kf-1 null mouse of the present invention may be usable as an effective means for elucidating various psychoneuroses, for example, anxiety disorder, decreased libido, social isolation, suicidal ideation and like depression-like behavioral abnormalities and symptoms of “social withdrawal disorder”. The kf-1 null mouse of the present invention may also be usable as an effective means for developing or screening a medical composition that can substantively in the prevention, treatment or alleviation of such symptoms.

### BRIEF DESCRIPTION OF THE DRAWINGS

FIG. 1 explains the gene targeting method used in the production of a kf-1 knockout mouse in the Example. The figure schematically shows the structures of, in order from the top, a kf-1 gene (wild-type allele) region in a normal chromosome, a targeting vector for the production of a kf-1 knockout mouse, a kf-1 gene that has undergone homologous recombination and into which a loxP site and a neomycin (G418) resistance gene have been inserted (homologous recombinant), and a mutant allele (Cre recombinant) from which a large portion of an kf-1 gene-translation region was deleted a result of a cross with a transgenic mouse expressing Cre.

FIG. 2 shows the results of the Southern blotting analysis of the genomic DNA isolated from a wild-type mouse, a homologous recombinant mouse, and a homozygous Cre recombinant mouse (kf-1(−/−)). The upper panel shows the structure of, from the top, the wild type kf-1 gene, recombinant kf-1 gene with a targeting vector and kf-1 mutant allele lacking exon 4 as a result of crossing with a Cre expressing transgenic mouse with the BspHI restriction sites, and the probe. The lower panel shows the results of Southern hybridization experiment conducted using the exon 4 of mouse kf-1 as a probe for BspHI-digested genomic DNAs derived from the wild-type mouse, a homozygote mouse with homologous recombination with the targeting vector, a heterozygote mouse having a kf-1 gene in which exon 4 is deleted in one of the alleles as a result of crossing with a Cre-expressing transgenic mouse, and a kf-1(−/−) homozygote mouse having no exon 4 in either allele of kf-1 gene. The lane 4 indicates that the exon 4 is completely lost in the kf-1 (−/−) mouse.

FIG. 3 shows the results of light/dark transition test in kf-1 (−/−) mice produced in Example 1. The wild-type mice (Controls) and kf-1 null mice (Mutants) were placed in a dark compartment at the beginning, and A: the distance traveled within the light compartment and the dark compartment, B: the stay time in the light compartment, C: the number of transitions between the light compartment and the dark compartment, and D: the latency time before the first entering into the light compartment were measured. The results of the significance test using a variance analysis indicated that mutant mice exhibited a significantly lower level of locomoter activity merely in the light compartment compared to that of the wild type (A) and remarkably shorter stay time in the light compartment than the wild-type mice did (B); however, there were no significant differences in the distance traveled between the two groups in the dark compartment. There was also no significant difference between the kf-1 null mice and the wild-type mice in the latency time spent until entering the light compartment for the first time.

From these results, it can be concluded that there is no significant difference between the mutant mice and the wild-type mice in general and exploratory locomoter activities; however, the mutant mice exhibited remarkably lowered locomoter activity in the light compartment. Because a significant difference was not observed in the time spent before entering the light compartment for the first time, it is clear that mutant mice are not suffering from remarked photophobia.

FIG. 4 shows the results of the elevated plus-maze test of kf-1 (−/−) mice that were produced in Example 1. The wild-type mice (Controls) and kf-1 null mice (Mutants) were placed individually at the center of the elevated plus-maze (center platform), and its activities were recorded for 10 minutes. This figure shows A: the number of entries into the center platform, B: the number of entries into the open arms, C: the total distance traveled, and D: percent of the stay time spent on the open arms. Mice inherently have acrophobia and tend to avoid the open arms. There was no significant difference between the wild-type mice and the mutant mice in this tendency (B, D). However, there were significant differences between the wild-type mice and the mutant mice in the frequency of entering the center of the plus-maze where the open arms cross with the closed arms (A), and the total distance traveled (C). It become clear that mutant mice tend to remain staying on either arm.

FIG. 5 shows the results of the forced swim test in kf-1 (−/−) mice that were produced in Example 1. The wild-type mice (Controls) and kf-1 null mice (Mutants) were placed individually in a water bath and their movements were recorded for 10 minutes. The graphs show the percent immobility time per minute (A), and the distance traveled every minute (B) (Day 1). The same test was repeated after 24 hours, and the results are shown as Day 2. In the forced swim test, there were no significant differences between the wild-type mice and the kf-1 (−/−) mice, and a lack of eagerness to live is not observed, which is an indication of the onset of “depression” symptoms in mice.

FIG. 6 shows the open-field-test results of the kf-1 (−/−) mouse produced in Example 1. The actions of wild-type mice (Controls) and kf-1 null mice (Mutants) on the open field were observed for 120 minutes, and total locomotion distance per 5 minute interval (A), count of vertical activity per 5 minute interval (B), the time spent in the center of the compartment per 5 minute interval (C), and count of stereotypic behaviors per 5 minute interval (D) was scored. There were no significant differences between the wild-type mice and the kf-1 (−/−) mice in their behavioral pattern. It is clear that a significant reduction of activity was not observed in the kf-1 null mice.

FIG. 7 shows the results of prepulse inhibition test in the kf-1 (−/−) mouse produced in Example 1. FIG. 7(A) shows that startle response amplitude of mutant mice is significantly reduced compared to that of the wild-type mice (Controls), which is shown as a ratio of the weight loaded upon startle response to the weight of a resting mouse at the time of acoustic stimulus without preceding stimulus (110 dB or 120 dB). This corresponds to an increase in their anxiety-like behaviors. FIG. 7(B) shows that percent prepulse inhibition was significantly increased in kf-1 (−/−) compared to that in the wild-type mice upon strong acoustic stimulus following the weak acoustic stimulus in comparison with that upon strong acoustic stimulus alone. Compared to the wild-type mice, the kf-1 (−/−) mice exhibited better learning effects after receiving the preceding stimulus. This indicates that the increased prepulse inhibition of the startle response corresponds to increased anxiety-like behaviors.

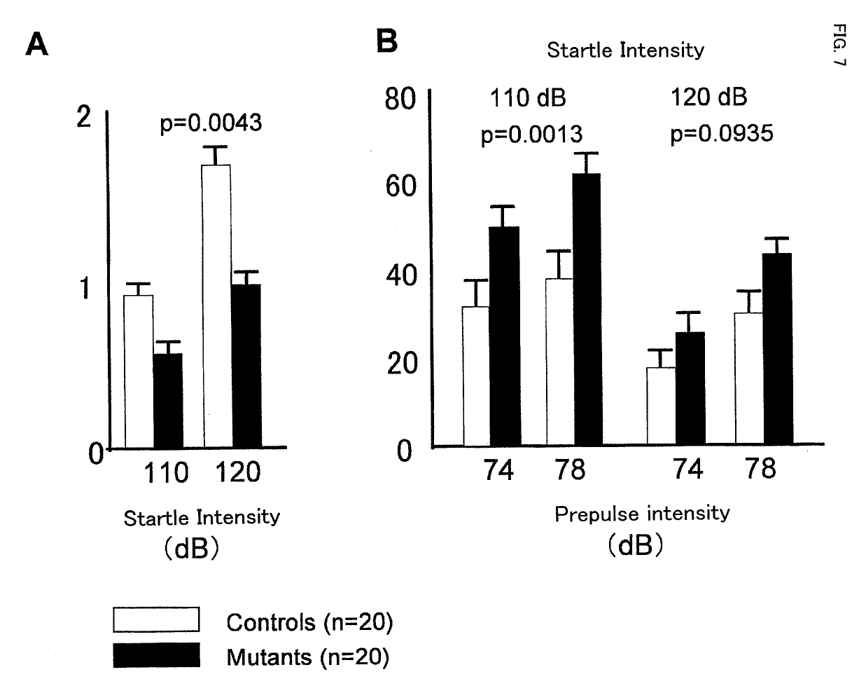

### BEST MODE FOR CARRYING OUT THE INVENTION

The Examples of the present invention are explained in detail with reference to the attached drawings. However, the scope of the present invention is not limited to these Examples.

### Examples

### 1. Creation of the kf-1 Knockout Mouse

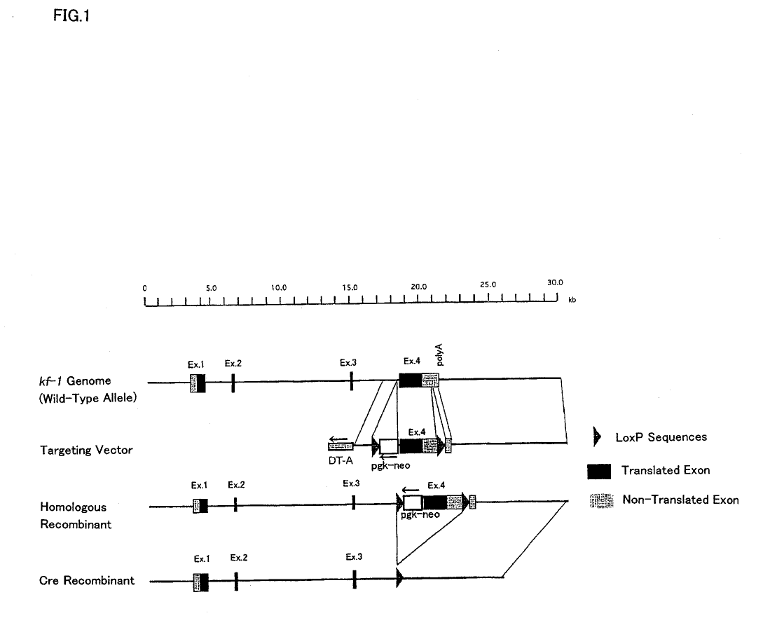

In order to construct the targeting vector, using mouse kf-1 cDNA shown by SEQ ID NO: 7 in the Sequence List as a probe, a mouse genomic DNA library was screened by plaque hybridization, and several genomic DNAs containing the mouse kf-1 gene were isolated. The mouse kf-1 gene consist of four exons ranging over approximately 20 Kb. By using the obtained mouse kf-1 gene DNA, a targeting vector was constructed as to line up from left to right, 1.9 Kb long AseI-StuI fragment located in the Intron 3 (Intron 3a) as the upstream arm, LoxP sequence, Neomycin resistant gene derived from pKJ2, 2.6 Kb long StuI-BglII fragment including Intron 3 (intron 3b) from the StuI site to exon 4 and the exon 4 from the beginning of it to the BglII site (exon 4a), LoxP sequence, and exon 4 from the BglII site to the polyA additional site (exon 4b) followed by the genomic downstream region as the downstream arm. A diphtheria toxin gene (DT-A) was placed in front of the upstream arm segment as a genetic marker for negative selection against clones without homologous recombination, and the resulting construct was used as the targeting vector (FIG. 1).

Note that pHSG396 (disclosed in Gene, 1987; 61:63-74.) was used for the backbone of this targeting vector.

Complete sequences of the thus-constructed targeting vector are shown by SEQ ID NO: 6 in the Sequence List.

Each region of the transgenic vector is explained below.

### Region Source

1-1206 pHSG396
1218-2772 pMC1_DTpA (diphtheria toxin gene)
2780-4695 mouse kf-1 gene intron 3a
4759-4942 pBS246 (LoxP cassette)
4997-6329 pKJ2 (pgk-Neo)
6376-6956 mouse kf-1 gene intron 3b
6957-8943 mouse kf-1 gene exon 4a
8959-9037 pBS246 (LoxP cassette)
9044-9074 mouse kf-1 gene exon 4b
9075-19222 mouse genome downstream

### 19223-19252 Linker

19562-20248 pHSG396.

The mouse kf-1 gene intron 3a had the sequence represented by SEQ ID NO: 1 in the Sequence Listing.

The mouse kf-1 gene intron 3b had the sequence represented by SEQ ID NO: 2 in the Sequence Listing.

The mouse kf-1 gene exon 4a had the sequence represented by SEQ ID NO: 3 in the Sequence Listing.

The mouse kf-1 gene exon 4b had the sequence represented by SEQ ID NO: 4 in the Sequence Listing.

The downstream region after mouse kf-1 gene has the sequence represented by SEQ ID NO: 5 in the Sequence Listing.

After linearization, the above-mentioned targeting vector was introduced into 129SVJ embryonic stem cells by electroporation. Clones resistant to antibiotic G418 were selected. Resulting 432 clones were subjected to PCR analysis using the two types of primers described below, and five homologous recombinant clones were obtained producing 483 bp long amplified fragment.

20039FW:
TCTTGGTTAAATAATGTATGCTCT
(the sequence represented by SEQ ID NO: 10)
17436FW:
AACTTGAAGTCGCTGTCTTTTGG
(the sequence represented by SEQ ID NO: 11)
20292RV:
CCCCTATAAAATTCTTTCCTATCC
(the sequence represented by SEQ ID NO: 12)

[1.3]

Chimeric mice were obtained by microinjecting the homologous recombinant ES clones into mouse early embryos by the known method (Genes Dev. (1994) 8, 707-719). The thus-obtained chimeric mice were crossed with wild-type C57BL/6 mice, and the resulting heterozygous male mice were crossed with Cre-expressing female mice, and then heterozygous mice with deletion of kf-1 Exon 4 were obtained. By crossing between male and female heterozygous mice, kf-1 knockout mice having homozygous deletion alleles (hereunder the mouse is referred to as a “kf-1(−/−) mouse”) were produced.

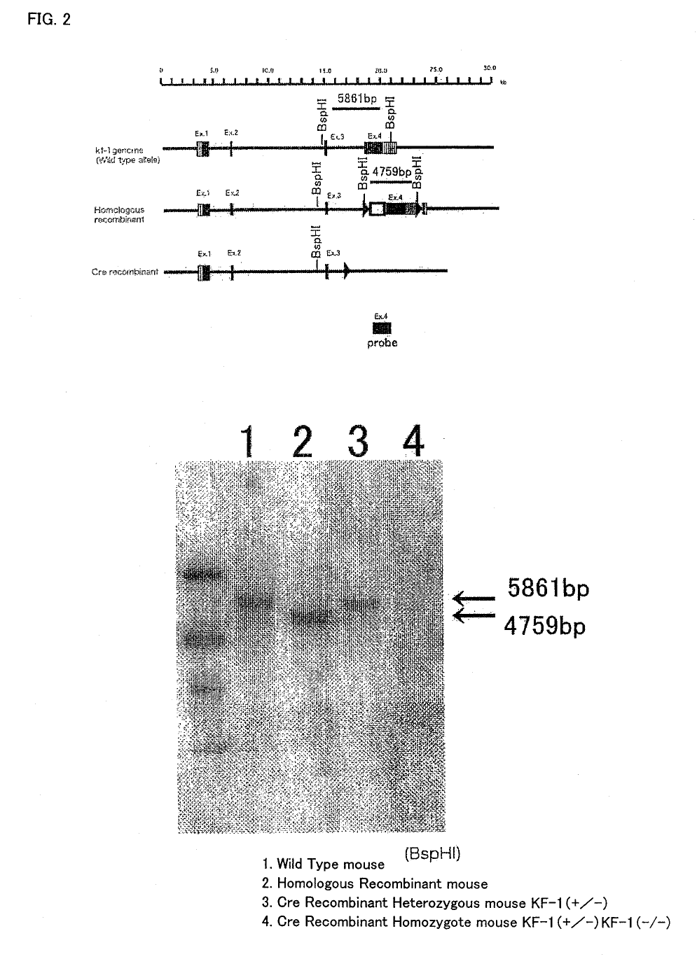

After having isolated genomic DNA from each mouse and having digested it with BspHI restriction enzyme, Southern hybridization was conducted using exon 4 of kf-1 gene as a probe. Consequently, a band of about 5.86 kb was observed in the wild type C57BL/6, and a band of about 4.76 kb was observed in the homologous recombinant mouse. In the Cre recombinant heterozygous mouse, the intensity of 5.86 Kb long DNA fragment decreased to a half of that obtained in the wild type, and no kf-1 Exon 4 bands were detected at all in the Cre recombinant homozygous mouse (kf-1 (−/−)) (FIG. 2).

Furthermore, using the primers shown below, PCR was conducted.

| | |
|---|---|
| 20039FW: | TCTTGGTTAAATAATGTATGCTCT |
| 17436FW: | AACTTGAAGTCGCTGTCTTTTGG |
| 20292RV: | CCCCTATAAAATTCTTTCCTATCC |

The results show that, the amplified product in the wild-type (WT) mouse was a 277 bp long fragment and the amplified product in the knockout mouse (Hicky) was a 560 bp long fragment.

There were no significant differences between the kf-1 (−/−) mouse and the wild-type mouse in appearance and reproductivity.

[1.4]

The obtained F1 kf-1(+/−) mice were repeatedly backcrossed in total 8 times to C57BL/6N. The resulting male and female kf-1(+/−) F9 mice were intercrossed with each other, and 20 male littermates of kf-1(+/+) and kf-1(−/−) (40 mice in total) were obtained. When the mice became four-weeks old, mice were group-housed with two kf-1(+/+) and two kf-1(−/−) littermates in one cage up to ten weeks old, and then used in the following behavioral experiments.

### 2. Behavioral Experiments of kf-1(−/−) Mice

The significant difference test was conducted using the variance analysis method. When p<0.05, it is judged that there is a significant difference.

### Light/Dark Transition Test

Whether or not mice display an increased anxiety was determined according to the anxiety disorder assessment method using light and dark boxes (Psychopharmacology, 94, 392-396, 1988). The experimental apparatus consists of two compartments, one is a dark compartment and the other is a light compartment. The light compartment was illuminated brightly with a lamp, and the dark compartment was connected next to the light compartment by tunnel. The test animal was placed first in the dark compartment and its behaviors were recorded for 10 minutes using a video camera installed in the lid of a box. The distance traveled within each box (Distance Traveled), the time spent in the light compartment (Stay Time in Light), the number of times traveled between the dark compartment and the light compartment (Transitions), and the time elapsed until the mouse enters the light compartment for the first time (Latency to Light) from the dark compartment were analyzed.

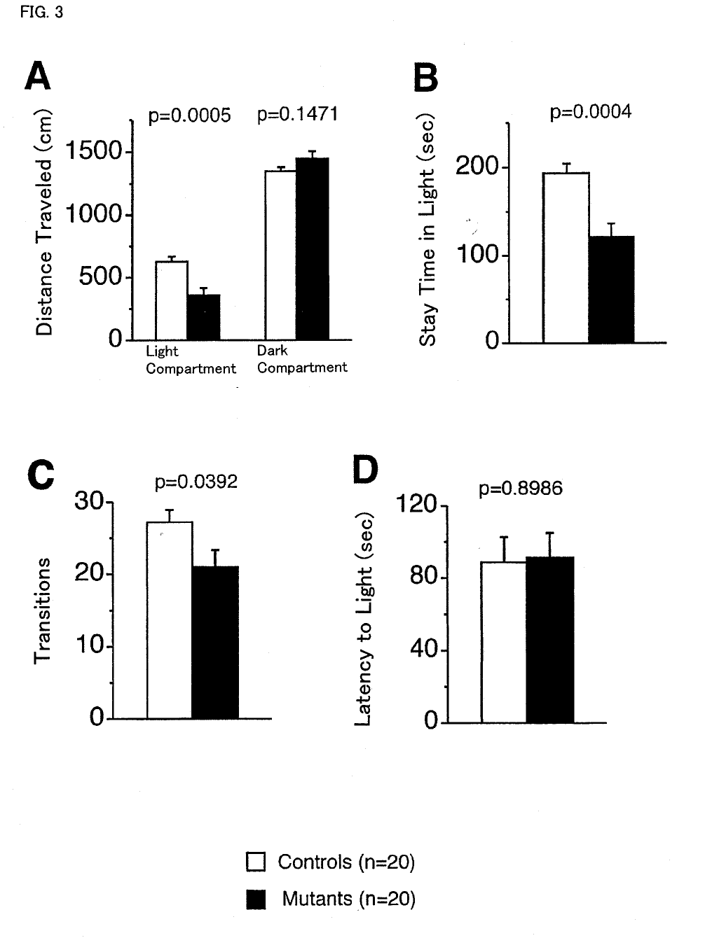

FIG. 3 shows the results.

The results show that there were no significant differences between the two groups in the total distance traveled within the dark compartment and the time elapsed before entering the light compartment from the dark compartment (Latency to Light).

However, in distance traveled in the light compartment, the time spent in the light compartment, and the number of transitions between the dark compartment and the light compartment, kf-1(−/−) mice showed significantly reduced outcomes compared to the control mice. In other words, increased anxiety was aroused in kf-1(−/−) mice compared with the control mice.

### [2.2] Elevated Plus-Maze Test

Rising anxiety in the kf-1(−/−) mouse was determined according to the anxiety-like behavior assessment method using an elevated plus-maze (Miyakawa T, et al., Proc Natl Acad Sci USA. 2003; 100:8987-8992.).

The plus-maze equipment has two open arms without walls and two closed arms with walls of the same size; and the plus maze is elevated. The arms are connected to a central square so as to obtain a cross-shaped maze. Each mouse was placed at the center platform and the monitoring was commenced. The following actions were recorded for 10 minutes: the number of entries into the center platform (Number of Entries), the frequency of entering into the open arms (Entries into Open Arms %), the total distance traveled (Distance Traveled) and the percentage of time spent in the open arms (Times on Open Arms).

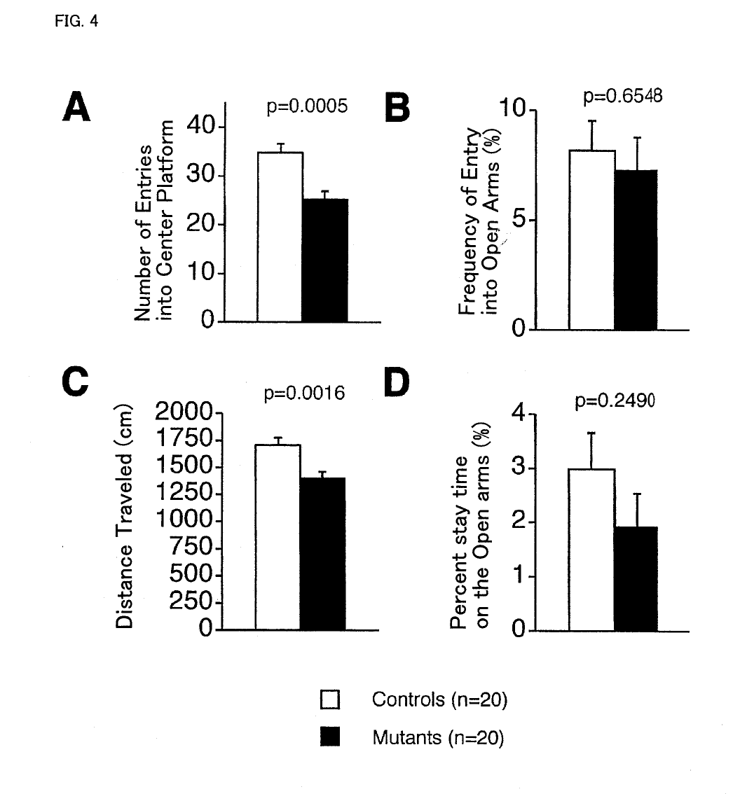

FIG. 4 shows the results.

The results showed that the number of entries into the center platform of the cross-shaped maze equipment and the total distance traveled were significantly reduced in kf-1(−/−) mice compared to the wild type mice. The number of entries onto open arms and stay time on the open arms were also reduced in mutant mice, but not significantly.

### [2.3] Forced Swim Test

Mouse “depression” symptoms were examined in terms of the lack of eagerness to live assessment, which is an indication of “depression” symptoms in mice by using a forced swim test (Arch. int. Pharmacodyn. 229, 327-336, 1977). The experimental equipment has four plastic cylindrical tanks with water therein. A mouse was placed in one of the tanks and its behaviors were recorded for 10 minutes. The analysis was conducted based on the result of measurements of immobility time per minute and the distance traveled per minute. The same experiment was repeated on the following day.

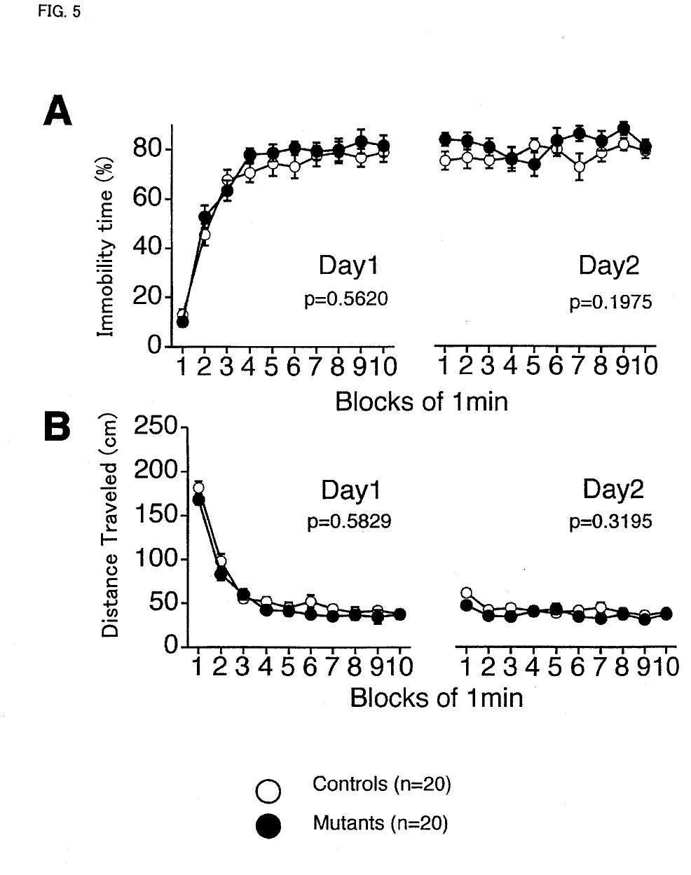

FIG. 5 shows the results.

The results show there was no significant difference between the kf-1(−/−) mice and the wild-type mice in terms of the immobility time and the distance traveled.

### [2.4] Open Field Test

Using the open field method, exploratory locomotion, general activity, and emotional behaviors of kf-1(−/−) mice were tested. The test animals were placed individually in a white acrylic cage, and the distance traveled (Total Distance), the number of times of rearing (Vertical Activity), the time spent in the central part (Center time), and the stereotypical behaviors (Stereotypic Counts) were measured, and changes in behaviors are shown at five-minute intervals on a graph.

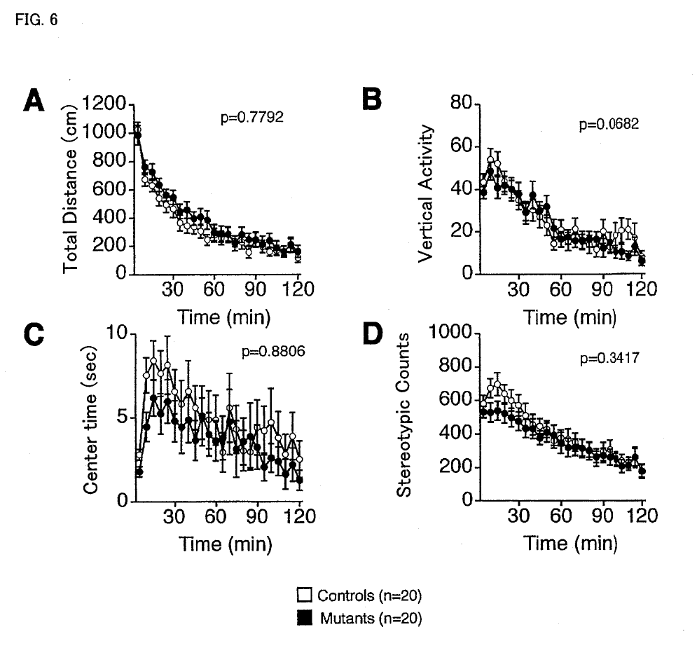

FIG. 6 shows the results.

There were no significant differences between the kf-1(−/−) mice and the control group mice in all categories.

### [2.5] Startle Response/Prepulse Inhibition Test

The prepulse inhibition test was used to detect dysfunction, if any, of sensorimotor gating, as observed usually in schizophrenic patients, in the kf-1(−/−) mice. When only a strong acoustic stimulus is given, the normal sensory information process is conducted as to produce a startle reaction. However, when a preceding weak acoustic stimulus is given in advance, the startle reaction is weakened. When the kf-1(−/−) mice were given strong acoustic stimulus (110 dB, 120 dB), they exhibited a significantly weaker startle reaction compared to the control mice. However, when a preceding weak acoustic stimulus (74 dB, 78 dB) was given in advance, they exhibited a significantly increased inhibition of the startle response than the control mice.

The analysis was conducted as described below. A mouse was placed in a test box for detecting a startle reaction equipped with a change of load measuring device. The mouse was allowed to adapt to the surroundings of the device for 10 minutes, and white noise background was presented in the box for 5 minutes to make the mouse adapt to the new atmosphere. The magnitudes of the startle reactions, when only strong acoustic stimulus (110 dB, 120 dB) was given, or when strong acoustic stimulus was given after giving preceding soft acoustic stimulus (74 dB, 78 dB) were expressed as an increase in load, i.e., A and B respectively. The reduction rate of the startle reaction (prepulse inhibition) C % can be obtained by the equation of C=100(1−B/A).

The results indicate that the kf-1(−/−) mice exhibited significantly lower startle reactions than the control mice when only strong acoustic stimulus was given. They also exhibited a significantly increased inhibition to startle reactions when strong acoustic stimulus was preceded by weak acoustic stimulus compared to the control mice. This may be interpreted that the kf-1(−/−) mice had a better learning ability, which is a reaction opposite to that observed in schizophrenia, and therefore it can be interpreted that the sensorimotor gating ability is augmented. The fact that the increased inhibition of startle response is associated with the enhancement of the anxiety-like behaviors in kf-1 (−/−) mice as observed in the elevated plus-maze test or the light/dark test indicates that depression and schizophrenia are caused by abnormalities of the same function, and the abnormality may be expressed in opposite directions, i.e., dysfunction or enhancement of the functional ability. This implies that the increase in anxiety-like behaviors may be detected by not only observing anxiety-like behaviors itself using the elevated plus-maze test and the light/dark test but also by measuring the suppression of the startle response using the prepulse inhibition test. The prepulse inhibition test can provide a simpler screening procedure as to anxiety-like behaviors.

### [2.6] Results Analysis

There were no significant differences observed between kf-1 null mice and the wild-type mice in body weight, the muscle strength test, etc. Furthermore, the results of the open-field test indicate that there was no significant difference in the general locomotor activity between the two groups. The results of the forced swim test show that so called mouse “depression” symptoms were not found in kf-1 null mice.

However, in the light/dark transition test and the elevated plus-maze test, it was confirmed that there were clearly significant differences between kf-1 null mice and the wild-type mice. The results of the elevated plus-maze test show that there was observed a significant decrease of exploratory locomotor activity on heights even though no significant differences were observed in acrophobia-like psychology. The results of the light/dark transition test indicate that the kf-1 null mice prefer to stay in the dark compartment and that the activity in the dark compartment was not reduced. It has been concluded that there is an association between the increased anxiety-like behaviors and the increased ability of sensorimotor gating function of kf-1 null mice observed in the prepulse inhibition test.

These behavioral patterns of kf-1 null mice suggested the possibility that kf-1 null mice could become a model animal for “social withdrawal disorder”.

### 3. Deposition of Organism

The above-obtained mouse was deposited with RIKEN, the Institute of Physical and Chemical Research, BioResource Center as Reg_No. 01916 (deposition date: Oct. 12, 2006, address of the deposit authority: 3-1-1, Koyadai, Tsukuba-shi, Ibaraki-ken, Japan).

The details of the deposited mouse are shown below.

Reg_No. 01916, systematic name: pKF1KO4.3.1, common-name and another strain name: Hicky mouse

### (1) Genetic Classification: Targeted Mutation Congenic

(2) Origin of the strain and Generation Number: A mouse was created from a mouse embryonic stem cell having LoxP sequences inserted before and after the exon 4 of a mouse kf-1 gene. The kf-1 exon 4 was deleted by crossing with Cre Tg mice. The passage number: 18 generations
(3) Microbiological Breeding Environment: conventional
(4) Microbiological Characteristics: ICLAS (microscopic examination I, culture I, serum I) negative, S. aureus positive Detail of Lineage (characteristics and use): Exhibiting “social withdrawal”-like anxiety behaviors. Usable as a model animal for developing novel anxiolytic agents and antidepressants
(5) Breeding and Mating: Propagation effectiveness A
(6) Development Process of Lineage: Mating with C57BL/6N, the eighth generation
(7) Remark: Abnormal behaviors were observed. Typical “social withdrawal disorder”-like symptoms and anxiety-like behaviors were observed in the light/dark test and the elevated plus-maze test. In the forced swim test and the tail suspension test, so called mouse “depression”-like behaviors were not observed.
(8) Name of the Introduced Vector: pKF1KO-4.3
(9) Introduced Gene: Lox-pgk-Neo-kf-1[ex4]-LoxP, more precisely LoxP-pgk-Neo-kf-1[in3b]-kf-1[ex4a]-LoxP
provided that the portion flanked by two LoxP cassettes, is deleted by crossing with Cre Tg mice, excepting a single LoxP site.
(10) Creation Method: ES cells

### (11) Name of ES Cells of (10): 129SVJ

### (12) Gene Detection Method: PCR

(13) Detail Of The Detection Method of (12) (primer sequence):

mgKF_U17436:
5′-AACTTGAAGTCGCTGTCTTTTGG
(the sequence represented by SEQ ID NO: 8),
Neo-F712:
5′-GAATGGGCTGACCGCTTCCTCGTG
(the sequence represented by SEQ ID NO: 9).

WT 277 bp, Hicky: 560 bp

1. A vector for creating a kf-1 gene knockout nonhuman animal exhibiting anxiety-like behaviors comprising: LoxP-pM-M-kf-1[in3b]-kf-1[ex4a]-LoxP, wherein, M is a selection marker gene,
pM is a promoter for expression of the selection marker gene,
kf-1[in3b] is a base sequence represented by SEQ ID NO: 2, and
kf-1[ex4a] is a base sequence represented by SEQ ID NO: 3.

2. A kf-1 gene knockout nonhuman animal created by using the vector of claim 1, or a descendant thereof.

3. A modified nonhuman animal or a descendant thereof of claim 2, or a descendant of the modified nonhuman animal.

4. (canceled)

5. (canceled)

6. A method for screening of compounds useful for prevention, treatment or alleviation of anxiety-like behaviors, the method comprising Steps (1) to (3) below:
(1) administering a test compound or placebo to the kf-1 gene knockout nonhuman animal created by using the vector of claim 1, or a descendant thereof;
(2) observing the anxiety-like behaviors of the nonhuman animal group to which the test compound is administered and that of the group to which the placebo is administered; and
(3) comparing the results in Step (2) to select a test compound that can suppress anxiety-like behaviors.

7. A method for creating a kf-1 gene knockout nonhuman animal, wherein the kf-1 gene knockout nonhuman animal exhibits significantly increased anxiety-like behaviors in at least one test selected from the group consisting of light-dark transition test, elevated plus maze test and startle response test, but does not exhibit significant differences in at least one test selected from the group consisting of the forced swim test and the tail suspension test in mice by comparing to the wild-type controls,
the method comprising following steps:
introducing the vector of claim 1 into a cell, and
obtaining a kf-1 gene knockout mouse created by using the cell.

8. A method for screening of compounds useful for prevention, treatment or alleviation of anxiety-like behaviors or anxiety in humans, wherein the anxiety-like behaviors or anxiety in humans is defined by exhibiting significantly increased anxiety-like behaviors in at least one test selected from the group consisting of light-dark transition test, elevated plus maze test and startle response test, but not exhibiting significant differences in at least one test selected from the group consisting of the forced swim test and the tail suspension test in mice by comparing to the wild-type controls,
the method comprising Steps (1) to (3) below:
(1) administering a test compound or placebo to the kf-1 gene knockout nonhuman animal created by using the vector of claim 1, or a descendant thereof:
(2) observing an anxiety-like behavior of the nonhuman animal group to which the test compound is administered and that of the group to which the placebo is administered; and
(3) comparing the results in Step (2) to select a test compound that can suppress an anxiety-like behavior.

9. A method for screening of compounds useful for prevention, treatment or alleviation of social withdrawal-like behaviors, the method comprising Steps (1) to (3) below:
(1) administering a test compound or placebo to the kf-1 gene knockout nonhuman animal created by using the vector of claim 1, or a descendant thereof;
(2) observing the social withdrawal-like behaviors of the nonhuman animal group to which the test compound is administered and that of the group to which the placebo is administered; and
(3) comparing the results in Step (2) to select a test compound that can suppress social withdrawal-like behaviors.
